(19) United States
(12) Patent Application Publication
(10) Pub. No.: US 2010/0167315 A1
(43) Pub. Date: Jul. 1, 2010

(54) METHOD FOR INVESTIGATING THE RESPONSE TO A TREATMENT WITH A MONOCLONAL ANTIBODY

(75) Inventors: Gilles Thibault, Ballan-Mire (FR); Hervé Watier, Ballan-Mire (FR); Sebastien Dall'Ozzo, Surrey (GB)

Correspondence Address: MORGAN LEWIS & BOCKIUS LLP, 1111 PENNSYLVANIA AVENUE NW, WASHINGTON, DC, 20004, US

(73) Assignees: GLYCODE, Uzarche (FR); UNIVERSITE FRANCOIS RABELAIS, Tours Cedex 1 (FR)

(21) Appl. No.: 12/441,237

(22) PCT Filed: Sep. 12, 2007
(86) PCT No.: PCT/IB2007/003498

§ 371 Date: Jan. 21, 2010

(30) Foreign Application Priority Data
Sep. 13, 2006 (FR) 0607990
Dec. 15, 2006 (EP) 06291942.8
Jan. 23, 2007 (FR) 0700456

(51) Int. Cl. G01N 33/567

(52) U.S. Cl. 435/7.21

(57) ABSTRACT

The invention relates to steps for the development or the quality control of recombinant monoclonal antibodies (MoAbr) used as medicaments, and also to the selection of the patients liable to effectively respond to a treatment with a given monoclonal antibody. More specifically, the invention relates to a method for evaluating, in vitro, the effector functions of NK cells in response to a test monoclonal antibody, comprising at least the following steps: (i) the NK cells are brought into contact with said monoclonal antibody, which is fixed on a support, in the presence of an agent for inhibiting the secretion of cytokines by said cells; (ii) by way of positive control for the activation of the NK cells, the same experiment is carried out using, in place of the test monoclonal antibody, a monoclonal antibody directed against the Fc RIIIa receptor; (iii) after an incubation period of at least 1 hour, the response of the NK cells is observed by measuring the presence of the CD107 marker at the cell surface, and also the presence of intracellular IFN and/or of intracellular TNF.

The present invention relates to the field of monoclonal antibodies (MoAbs), in particular that of recombinant monoclonal antibodies (rMoAbs) used as medicaments. More specifically, the invention relates to the steps for the development or the quality control of these antibodies, and also to the selection of the patients liable to effectively respond to a treatment with a given monoclonal antibody.

The bioengineering of rMoAbs has enabled the development of medicaments such as rituximab, infliximab or trastuzumab, targeting CD20, TNFα and the ErbB2 antigen, respectively, and which constitute major therapeutic advances in particular in cancerology and in systemic inflammatory diseases. However, although they are designed on the model of antibodies naturally produced by the organism, a considerable variability in therapeutic response and in adverse effects exists between patients. A part of the variability in response is not related to the disease itself (cancer, rheumatoid arthritis, Crohn's disease, etc.), but to individual factors, such as the genetic make-up of the patient. It is therefore important to analyze the mechanisms involved in the response to a MoAb, in order to optimize these treatments.

An antibody (Ab) is a bifunctional molecule constituted of a Fab portion for binding to the antigen (Ag) for which it is specific, and of an Fc portion capable of recruiting, after binding to the Ag, active molecules (complement system) or cells expressing receptors (FcRs) for this portion. These molecules and cells then perform effector functions such as complement-dependent cytotoxicity (CDC) or antibody-dependent cellular cytotoxicity (ADCC), which make it possible to eliminate the Ag or the cell expressing the Ag (desired effect in anticancer treatments, for example). The binding of the Ab to the Ag, which is independent of its Fc portion, may be sufficient to neutralize some of them, or even to trigger apoptosis of the cell which expresses it.

Therapeutic rMoAbs can act by means of these various mechanisms, but the relative part of each of them is most commonly poorly understood. Studies carried out in mice have made it possible to show that mechanisms dependent on the interaction of the Fc portion of the rMoAb with the FcRs are involved in the activity of cytolytic (cell-destroying) rMoAbs in vivo. It has also been shown that the clinical response of patients suffering from non-Hodgkin's malignant lymphomas, to rituximab, is dependent on the polymorphism of the FCGR3A gene (Cartron et al., 2002). This gene encodes FcγRIIIa (also known as CD16a), an FcR having low affinity for immunoglobulins (Ig) G, expressed mainly on natural killer (NK) cells and macrophages, which are responsible for ADCC. The polymorphism is reflected by the presence of a valine (V) or a phenylalanine (F) at position 158 of FcγRIIIa. The influence of the 158-V/F polymorphism has been confirmed by other teams for this same rMoAb, including in other malignant hemopathies, but also for other rMoAbs. Dall'Ozzo et al. have also demonstrated that the ability of NK cells to exercise ADCC is dependent on the polymorphism of FCGR3A. This ability is greater when the cells express only the 158-V allotype which has a better affinity for the Fc of the rMoAbs (Dall'Ozzo et al., 2004). These results demonstrate that mechanisms of action dependent on FcγRIIIa and on cells expressing this receptor, in particular NK cells, are strongly involved in the therapeutic activity of these rMoAbs in humans.

NK cells are part of the class of large granular lymphocytes (LGLs). They participate in the immunological defense system which is nonspecific, with respect to tumor cells, viruses, bacteria which develop intracellularly, and allogenic cells (Trinchieri, 1989). These cells, generally identified by the membrane expression of CD56 and the absence of expression of the CD3 molecule, represent 5 to 20% of the lymphocytes. As their name indicates, they exercise a cytotoxic activity without prior immunization. This cytotoxicity, termed natural cytotoxicity, is triggered by the stimulation of activating receptors, the ligands of which are expressed on target cells having undergone stress, such as a tumoral transformation or infection with a virus. As mentioned above, NK cells also exercise ADCC, which is triggered by the engagement of FcγRIIIa/CD16a (expressed by approximately 95% of them) by the Fc portion of the antibodies specific for the target cell and bound to the latter. The involvement of ADCC in the mechanisms of action of cytolytic therapeutic rMoAbs is now well established (Dall'Ozzo et al., 2004; Greenwood et al., 1993; Reff et al., 1994). These two triggering mechanisms result in activation of the NK cell, and then in degranulation thereof, and thus in the release of the cytolytic content thereof: mainly composed of granzymes and perforin, which have a protease activity and pro-apoptotic activity on the target cells (Smyth et al., 2005). These granules also contain, at their membrane, highly N-glycosylated glycoproteins which are part of the family of LAMPs (lysosome associated membrane proteins), which comprise LAMP-1 (CD107a), LAMP-2 (CD107b) and LAMP-3 (CD63) (Fukuda, 1991), which are not expressed on the plasma membrane. During the degranulation phenomenon, the effector cells release their perforin and the LAMP molecules are concomitantly redistributed on the plasma membrane of the cytotoxic cell (Betts et al., 2003). They then become accessible to labeling with fluorescent MoAbs detectable by flow cytometry. The membrane expression of CD107 thus makes it possible to identify the cells having undergone degranulation (Betts et al., 2003) and exerted cytotoxicity (Bryceson et al., 2005). The detection of CD107 can therefore represent an alternative to the conventional cytotoxicity tests based on the release, by the lyzed target cells, of labels, such as ⁵¹Cr, with which they have been previously loaded (Brunner et al., 1968).

NK cells also produce cytokines with the two triggering mechanisms described above: natural or Ab-dependent (antibody-dependent cytokine production: ADCP). This secretion is composed mainly of interferon gamma (IFNγ), tumor necrosis factor (tumor necrosis factor alpha: TNFα) and GM-CSF (Trinchieri, 1989). This cytokine production participates in the recruitment of the other inflammation mediators at sites of infection or of tumor growth. IFNγ enables macrophage recruitment, increased expression of MHC class I and class II molecules, development of the specific immune response, and synthesis of other cytokines which influence the orientation of T lymphocytes toward a Th1-type response (Boehm et al., 1997; Gajewski et al., 1988; Nathan et al., 1984). Various hypotheses have been proposed to explain the mechanisms which orient NK cells toward cytotoxicity or cytokine secretion triggered according to the natural mechanism: the existence of two distinct NK subpopulations (Cooper et al., 2001), the nature of the target cell (Kurago et al., 1998), the stimulation of cell coreceptors (Colucci et al., 2001; Rajagopalan et al., 2001). These results are to be related to those reported by Faroudi et al., showing the cytokine response of CD8+ T lymphocytes requires an activation threshold that is above that of the cytotoxicity (Faroudi et al., 2003).

ADCP has been studied relatively little. Anegon et al. have shown that the stimulation of NK cells with an anti-CD16 leads to the accumulation of IFNγ mRNA and TNFα mRNA (Anegon et al., 1988). However, the secretion of the corresponding cytokines usually requires costimulation of the NK cells by cytokines such as IL-2, IL-12 and IL-15 (Colucci et al., 2001; Huntington et al., 2005; Parihar et al., 2002). Similarly, NK cells placed in the presence of SKBR-3 target cells sensitized with trastuzumab, a therapeutic MoAb directed against the ErbB2 antigen expressed by these cells, perform ADCP only in the presence of IL-2 or of IL-12 (Parihar et al., 2002), whereas they perform ADCC in the absence of these cytokines (Cooley et al., 1999). The two functions are therefore not always regulated, in vitro, in a coordinated manner. It has been proposed that, in vivo, the engagement of FcγRIIIa by therapeutic MoAbs might trigger a cytokine response (via ADCP) which contributes to their pharmacological responses (Parihar et al., 2002; Wing et al., 1996) or to adverse effects such as the cytokine release syndrome observed during the first infusion (Wing et al., 1996).

At the current time, it appears to be necessary to improve the effectiveness of treatments with rMoAbs. Several approaches can be envisioned for this.

The first approach consists in attempting to optimize or modify the medicament itself. It is thus possible to generate rMoAbs which have a better affinity for the target Ag, to generate rMoAbs directed against a more relevant epitope or to modify the Fc portion of the rMoAbs in such a way as to increase the effectiveness of the functions dependent thereon. Variations in the glycosylation of the Fc portion (by having these rMoAbs produced by particular cells) or modifications to the protein sequence have thus made it possible to increase the affinity of rMoAbs for soluble forms of FcγR and, from a functional point of view, to increase the ADCC exerted in vitro in the presence of these modified rMoAbs on given target cells expressing the Ag (Shields et al., 2001; Shinkawa et al., 2003; Umana et al., 1999). Other modifications can also result in complement activation being improved. These rMoAbs having a modified Fc region (second-generation rMoAbs) are currently in the development phase. Another way to improve treatments with antibodies would be to administer adjuvant therapeutics for increasing the FcγRIIIa-dependent responses, as has already been done with cytokines such as IL-2 or IL-12. Tools for readily testing the effect of the modifications introduced into the Fc region and/or of the introduction of an adjuvant, on the activation of the effector cells, would be very useful in this context. Similarly, such tools would make it possible to test the (inter-batch) variability and/or the (intra-batch) stability of the functional properties of the Fc portion in the rMoAb production phase.

Another approach for improving the effectiveness of treatments with rMoAbs consists in optimizing the selection of the patients liable to receive the treatments. The identification of the genetic therapeutic-response factors, such as the polymorphism of FCGR3A, which is the field of pharmacogenetics, may make it possible to perform a better selection of the responding patients, but may also make it possible to optimize the treatment in the “non-responders”, in particular by modification of administration schemes, addition of synergic treatments, etc. However, to date, genetics makes it possible to categorize patients only in statistically good-responder or poor-responder groups. Thus, among the patients belonging to the poor-responder genetic group, some show a favorable therapeutic response. Consequently, the use of tests for evaluating the individual quality of response of the FcγRIIIa+ effector cells of patients could be more predictive of the therapeutic response.

The present invention proposes tools that can be used in the three applications mentioned above (improvement and quality control of therapeutic rMoAbs and patient selection), since it concerns a technology which is simple to implement, reproducible and reliable, for quantifying the effector functions of cells expressing FcγRIIIa (ADCC/ADCP balance), in response to rMoAbs. This technology is based on the combination of several means, and on the demonstration that it is possible to simultaneously analyze the ADCC and ADCP responses of effector cells expressing FcγRIIIa (in particular NK cells), by incubating these cells in the presence of rMoAbs fixed on a plastic support, which thus bind the FcγRIIIa, while being free of any cell target. It thus becomes possible to compare the ability of different rMoAbs (recognizing various Ags on various targets) to induce ADCC and ADCP by NK cells or other cell effectors.

The invention therefore relates, firstly, to a method for evaluating, in vitro, the effector functions of cells expressing FcγRIIIa, in particular of NK cells, in response to a test monoclonal antibody, comprising at least the following steps:

(i) the cells expressing FcγRIIIa are brought into contact with said monoclonal antibody, which is fixed on a support, in the presence of an agent for inhibiting the secretion of cytokines by said cells;
(ii) by way of positive control for the activation of the cells expressing FcγRIIIa, the same experiment is carried out using, in place of the test monoclonal antibody, a monoclonal antibody directed against the FcγRIIIa receptor;
(iii) after an incubation period of at least 1 hour, the response of the cells expressing FcγRIIIa is observed by measuring the presence of the CD107 marker at the cell surface, and also the presence of intracellular IFNγ and/or of intracellular TNFα.

In the above method, the presence of the CD107 marker at the cell surface, and the intracellular IFNγ and/or the intracellular TNFα, are preferably measured by flow cytometry.

According to a preferred implementation of the method of the invention, the incubation is carried out in the presence of an anti-CD107 monoclonal antibody coupled to a label. This will make it possible to measure, at the end of the incubation, the presence of the CD107 marker at the cell surface, which reveals the cells having degranulated.

In one method according to the invention, the measurement of the IFNγ and/or of the TNFα is preferably carried out by permeabilizing the cells at the end of the incubation, and then by using a monoclonal antibody directed against the cytokine to be measured, coupled to a label.

The inventors have, moreover, confirmed that the engagement of FcγRIIIa (CD16) by an antibody is reflected by a loss of membrane expression of this molecule on the cells. The detection of CD16 at the surface of the cells thus provides additional information on the response of the cells to a given rMoAb. According to another preferred implementation of the present invention, the above method therefore also comprises a step of measuring the expression of CD16 at the surface of the cells expressing FcγRIIIa, by flow cytometry after labeling said cells, at the end of the incubation, with an anti-CD16 monoclonal antibody coupled to a label.

The flow cytometry technique is well known to those skilled in the art, who will be able to choose suitable labels for distinguishing the various molecules to be measured. In particular, it is advantageous to choose, as labels coupled to the anti-CD107, anti-IFNγ, anti-TNFα and anti-CD16 monoclonal antibodies, fluorochromes which emit at distinct wavelengths. By way of nonlimiting examples, mention may be made of fluorescein isothiocyanate (FITC), Alexa Fluor 488, Alexa Fluor 405, phycoerythrin (PE), peridinin chlorophyll protein (PerCP), phycoerythrin cyanin 5 (PC5), phycoerythrin cyanin 5.5 (PC5.5), phycoerythrin Taxas Red® (ECD), phycoerythrin cyanin 7 (PC7), allophycocyanin (APC), Alexa Fluor 647, Alex Fluor 700, phycoerythrin Alexa Fluor 700 (PAF7), Alexa Fluor 700 and APC cyanin 7 (APC7).

Similarly, those skilled in the art can choose any agent suitable for inhibiting the secretion of cytokines by the cell expressing FcγRIIIa, from the compounds known to inhibit exocytosis, such as, for example, Brefeldin A.

The test monoclonal antibody and the monoclonal antibody directed against the FcγRIIIa receptor used as control are preferably attached at a concentration which makes it possible to saturate the support onto which they are adsorbed. Plates that can be used for implementing the method of the invention can in particular be obtained by sensitizing them with a concentration of approximately 1 to 3 μg/ml of antibody, for at least 6 h, preferably 8 to 12 h or more. However, in certain situations, it may be preferable to use subsaturating concentrations of antibody, for example for determining the sensitizing concentration of a given rMoAb which makes it possible to obtain a measurable effector response of the cells expressing FcγRIIIa. In this case, the plates will be rather obtained by sensitizing them with a concentration of approximately 0.01 to 1 μg/ml of antibody, for the same time as indicated above.

In order to allow activation of the cells expressing FcγRIIIa, the incubation of these cells with the monoclonal antibodies should preferably last at least 1 h, preferably 2 to 4 hours, before the analysis of the various response markers.

In one particular implementation of the method according to the invention, a step (iia) of stimulating the cells expressing FcγRIIIa, with a mixture of a phorbol ester (for example, phorbol 12-myristate 13-acetate (PMA)) and of a calcium ionophore (CaI) (for example, the calcium ionophore A23187), is added by way of positive control for the (overall) response capacity of said cells. This step is particularly useful when the method is used to carry out a diagnosis predictive of the response to a treatment with monoclonal antibodies, for a patient at an advanced stage of a disease involving the immune system (such as HIV infection).

At the end of the incubation, the method of the invention may advantageously comprise the following sequence:

(iv) the cells expressing FcγRIIIa are labeled with an anti-CD16 antibody coupled to a label,
(v) the cells expressing FcγRIIIa are permeabilized, and then labeled with an anti-IFNγ monoclonal antibody coupled to a label, and/or with an anti-TNFα monoclonal antibody coupled to a label,
(vi) the cells thus labeled are analyzed by flow cytometry.

According to one variant intended to refine the information collected on the activation of NK cells by a monoclonal antibody, the method described above can be modified by costimulating the NK cells, in steps (i) and (ii), with antibodies directed against one or more of the membrane receptors chosen from the following nonexhaustive list: NKp30, NKp46, NKG2D, DNAM (CD226), CD2, CD27, 2B4 (CD244), CD11a, CD11b, CD45 and CD160 (in addition to the stimulation with, on the one hand, the test monoclonal antibody and with, on the other hand, the anti-FcγRIIIa antibody used by way of control). This costimulation can be carried out by adsorbing, onto the same support as the test antibodies (or control antibodies), monoclonal antibodies directed against the receptor(s) in question.

According to the objective with which it is carried out, the method of the invention may use various sources and levels of purification of cells expressing FcγRIIIa. For example, it may involve a cell line (NK, or T lymphocytes), or NK cells or T lymphocytes isolated from one or more blood samples (for example, NK cells purified using a protocol as described in the experimental section below). The above method may also be applied to monocytes, but less advantageously. The cells may also be cells expressing FcγRIIIa after transfection of the FCGR3A gene (modified or non-modified) and possibly in combination with the FCERIG gene encoding the transducing molecule FcR-γ. When the invention is implemented on cells originating from blood samples, these cells may either be purified, or be present in fractions comprising several cell types, such as peripheral blood mononuclear cells (PBMCs) or peripheral blood lymphocytes (PBLs); advantageously, the method of the invention may also be implemented on a whole blood sample, originating from healthy donors or from patients. When the cells used have not been completely purified, for example when the method is implemented on whole blood samples, the cells are preferably also labeled with an anti-CD56 monoclonal antibody coupled to a label and, where appropriate, with an anti-CD3 monoclonal antibody and/or an anti-CD 14 monoclonal antibody coupled to another label, before the step of analysis by flow cytometry. This labeling, preferably carried out before the permeabilization of the cells, makes it possible to distinguish the various cell types of the sample (CD56+CD3− NK cells, CD3+ T lymphocytes, CD14+ monocytes, etc.).

A first application of the method described above in its various variants is to carry out a diagnostic test predictive of the response of an individual to a treatment with a therapeutic monoclonal antibody. In this case, the method is carried out with, of course, the antibody (or the antibodies) that it is envisioned to administer to the patient, and cells expressing FcγRIIIa originating from the patient. These cells may be purified (for example, NK cells isolated from a blood sample) or not purified (the method is then carried out on a whole blood sample from the patient).

The method according to the invention may also be used for quantifying the effector functions of the cells expressing FcγRIIIa in response to the monoclonal antibodies, or for comparing the ability of various recombinant monoclonal antibodies to induce a decrease in expression of FcγRIIIa, antibody-dependent cellular cytotoxicity (ADCC) and antibody-dependent cytokine production (ADCP) by cells expressing FcγRIIIa, in particular NK cells.

As mentioned above, the methods of the invention constitute an effective research tool for improving the effectiveness of recombinant monoclonal antibodies for therapeutic purposes, for example for testing the effects of modifications of the Fc portion thereof and/or the effect of various adjuvants, such as IL-2 or IL-12.

The present invention also provides an alternative method to that currently used by the European Pharmacopeia for performing a quality control of the functions of the Fc portion of immunoglobulins (Igs) administered intravenously (IVIgs). The technology currently used to evaluate these functions is in fact based on complement activation and not on FcR recruitment, which limits the advantage of its possible use in the rMoAb sector. There exists, today, a need for a reference technology for evaluating the effector functions of FcR-dependent rMoAbs, and particularly with respect to an FcR of which the clinical involvement is demonstrated, which is unquestionably the case of FcγRIIIa, and independent of the antigen targeted. The method of the invention can therefore be advantageously used for this.

The present invention also relates to a kit that can be used for implementing the method according to the present invention, comprising at least the following elements:
an anti-CD16 monoclonal antibody coupled to a label,
an anti-IFNγ monoclonal antibody coupled to a label and/or an anti-TNFα monoclonal antibody coupled to a label, and
a plate comprising at least one well coated with a monoclonal antibody directed against the FcγRIIIa receptor.

Those skilled in the art will be able to add to this kit so as to allow the implementation of the various variants of the method, described above.

The present invention is illustrated by means of the figures and the experimental description which follows.

### FIGURE LEGENDS

FIG. 1: ADCC and NK-cell degranulation after incubation with rituximab-sensitized Daudi cells. Purified NK cells were incubated with Daudi cells labeled with ⁵¹Cr (A) or with unlabeled Daudi cells in the presence of an anti-CD107-PC5 MoAb (B) at the effector-to-target (E:T) ratios indicated, in the presence of 2 μg/ml of rituximab, for 4 h at 37° C. A) The radioactivity was measured in the supernatants and the lysis percentages were calculated according to the formula indicated in the materials and methods. B) The evaluation of the percentage of CD107⁺ NK cells was carried out by flow cytometry, after labeling with an anti-CD56-PE MoAb (B). C) The percentage lysis was expressed relative to the total number of CD107% NK cells (% of CD56+CD107+ cells×number of NK cells incubated at the E:T ratio considered).

FIG. 2: ADCP and synthesis of IFNγ by the NK cells after incubation with rituximab-sensitized Daudi cells. Purified NK cells were incubated with Daudi cells at the E:T ratios indicated, in the presence of 2 μg/ml of rituximab and in the absence (A) or in the presence (B) of brefeldin A used to block the secretion for 4 h at 37° C. A) The concentration of IFNγ in the supernatant was measured by the ELISA method. B) The evaluation of the percentage of IFNγ+NK cells was carried out by flow cytometry, after labeling with an anti-CD56-PC5 MoAb, and then permeabilization and labeling with an anti-IFNγ-PE MoAb. C) The concentration of IFNγ in the supernatant was expressed as a function of the total number of IFNγ+NK cells (% of CD56+ IFNγ+ cells×number of NK cells incubated at the E:T ratio considered).

FIG. 3: Influence of brefeldin A on NK cell degranulation. Purified NK cells were incubated with Daudi cells in the presence of an anti-CD107-PC5 MoAb at the E:T ratios indicated, in the presence of 2 μg/ml of rituximab and in the absence (white bars) or in the presence (dotted bars) of brefeldin A used to block the secretion for 4 h at 37° C. The evaluation of the percentage of CD107+NK cells was carried out by flow cytometry, after labeling with an anti-CD56-PE MoAb.

FIG. 4: Degranulation and synthesis of IFNγ by NK cells after incubation with Daudi cells sensitized with rituximab or the anti-CD16 MoAb 3G8. Purified NK cells were incubated with Daudi cells in the presence of an anti-CD107-PC5 MoAb at the E:T ratio of 0.5:1, in the presence of brefeldin A and in the absence (A) or the presence of 2 μg/ml of rituximab (B) or of 2 μg/ml of the anti-CD16 MoAb 3G8 (C) for 4 h at 37° C. The cells were then labeled with an anti-CD56 MoAb, and then permeabilized, labeled with an anti-IFNγ-PE MoAb and analyzed by flow cytometry. The cytograms represent the expression of CD107 (along the y-axis) and of intracellular IFNγ (along the x-axis) of the CD56+ cells.

FIG. 5: Degranulation and synthesis of IFNγ by NK cells stimulated with PMA and CaI. Purified NK cells were incubated with the indicated concentrations of PMA and/or of CaI in the presence of an anti-CD107-PC5 MoAb and in the presence of brefeldin A for 4 h at 37° C. The cells were then permeabilized and labeled with an anti-IFNγ-PE MoAb and analyzed by flow cytometry. For each stimulation condition, the percentages of CD107-IFNγ+NK cells are represented in gray, those of CD107+IFNγ− NK cells are represented in white with dots and those of doubly positive cells (CD107+IFNγ+) in gray with dots.

FIG. 6: Kinetics of degranulation and of IFNγ synthesis by NK cells stimulated with PMA and CaI. Purified NK cells were treated as described in the legend of FIG. 5 with the exception of the incubation time, which was between 1 h and 4 h. The percentages of IFNγ+NK cells are represented in gray and those of CD107+NK cells are represented in white with dots.

FIG. 7: Expression of CD16, degranulation and synthesis of IFNγ by NK cells after incubation in plates sensitized with the anti-CD16 MoAb 3G8. The plates were sensitized for 12 h with the indicated concentrations of anti-CD16 MoAb 3G8, the binding of which was revealed by an ELISA technique using a peroxidase-coupled anti-murine IgG Ab. The curve represents the absorbances (y-axis on the left) as a function of the concentration of 3G8 used for sensitizing the plate (x-axis). Purified NK cells were incubated in plates sensitized with the anti-CD16 MoAb 3G8 in the presence of an anti-CD107-PC5 MoAb and in the presence of brefeldin A for 4 h at 37° C. The cells were then labeled with an anti-CD16-FITC MoAb, permeabilized, labeled with an anti-IFNγ-PE MoAb and analyzed by flow cytometry. The percentages of positive cells (y-axis on the right) are represented as a function of the sensitizing concentration of 3G8. The percentages of CD16+NK cells are hatched; those of CD107+IFNγ−, CD107−IFNγ+ and CD107+IFNγ+NK cells are represented as in FIG. 5.

FIG. 8: Adsorption of therapeutic rMoAbs and of human IgGs onto the plates. The plates were sensitized with the indicated concentrations of each rMoAb or of each human IgG subclass overnight at 4° C. The amount of rMoAb or IgG adsorbed was determined by means of an ELISA assay using a peroxidase-coupled anti-Fc antibody. The absorbance values represented correspond to the difference in the absorbances measured at 492 and 620 nm.

FIG. 9: Binding of C1q to the therapeutic rMoAbs and to the human IgGs adsorbed onto the plates. The plates were sensitized with 5 μg/ml of each of the rMoAbs or of entanercept or of polyvalent IgG or of human IgG1 or of human IgG3 or of the anti-CD16 MoAb 3G8, overnight at 4° C. The plates were then incubated with the indicated concentrations of human C1q. The amount of C1q bound to the antibodies was then measured using a peroxidase-coupled anti-C1q antibody. The absorbance values represented correspond to the difference in the absorbances measured at 492 and 620 nm.

FIG. 10: Compared affinity of the rMoAbs for the FcγRIIIa of NK cells. The NK cells of a donor (of genotype 158-FF) were purified, and incubated with the indicated concentrations of each of the rMoAbs or of polyvalent IgGs, and then with a suboptimal concentration (0.1 μg/ml) of the anti-CD16 MoAb 3G8 conjugated to FITC. The percentage inhibition of the binding of 3G8-FITC (y-axis) for each of the products tested was calculated according to the formula described in the “materials and methods” section.

FIG. 11: Loss of CD16 expression, degranulation and IFNγ synthesis by NK cells after incubation in sensitized plates: comparison of the mean responses induced by the therapeutic rMoAbs, the human IgGs or the anti-CD16 MoAb 3G8. The plates were sensitized with 5 μg/ml of each of the rMoAbs or of entanercept or of polyvalent IgG or of human IgG1 or of human IgG3 or of anti-lymphocyte serum or of the anti-CD16 MoAb 3G8, overnight at 4° C. The purified NK cells were incubated and analyzed as described in FIG. 7. The percentages of NK cells having lost CD16 expression are represented as hatched. Those of CD107−IFNγ+, CD107+IFNγ− and CD107+IFNγ+NK cells are represented as in FIG. 5. The results are expressed as mean±standard deviation and were obtained over the course of 8 experiments.

FIG. 12: Loss of CD16 expression, degranulation and IFNγ synthesis by NK cells after incubation in sensitized plates: comparison of the responses of the NK cells of 3 donors induced with the therapeutic rMoAbs, the human IgGs or the anti-CD16 MoAb 3G8. The individual results obtained with the NK cells of 3 of the donors (A, B and C) of FIG. 11 are represented.

FIG. 13: Degranulation and IFNγ synthesis by purified NK cells or by CD56+PBMCs after incubation in plates sensitized with 3 therapeutic rMoAbs, or the anti-CD16 MoAb 3G8. The plates were or were not sensitized with 5 μg/ml of each of the therapeutic rMoAbs or of the anti-CD16 MoAb 3G8, overnight at 4° C. The PBMCs or the purified NK cells of the same donor were incubated in the sensitized plates in the presence of an anti-CD107-PC5 MoAb and in the presence of brefeldin A, for 4 h at 37° C. The cells were then labeled with an anti-CD56-FITC MoAb, permeabilized and labeled with an anti-IFNγ-PE MoAb and analyzed by flow cytometry. The percentages of CD107+IFNγ+, CD107+IFNγ− and CD107+IFNγ+NK cells are represented as in FIG. 5.

FIG. 14: CD16 expression, degranulation and IFNγ synthesis by NK cells after incubation in plates cosensitized with the anti-CD16 MoAb 3G8 or infliximab and with MoAbs directed against the NK cell coactivating molecules. The plates were sensitized for 12H with or without 1.5 μg/ml, final concentration, of anti-CD16 MoAb or of infliximab, in combination or not in combination with 0, 5 or 5 μg/ml, final concentration, of murine IgG1 kappa (IgG1k) used as negative control, or of one of the MoAbs indicated which are directed against the NK cell activating or coactivating molecules. The purified NK cells were incubated and analyzed as described in FIG. 7. The hatched, dotted and gray histograms represent, respectively, the percentages of CD16+, CD107+ and IFNγ+ cells obtained after incubation (A) in plates sensitized, respectively, with only the MoAbs directed against the activating or coactivating molecules, (B) with these MoAbs and with the anti-CD16 MoAb 3G8 and (C) with these MoAbs and with infliximab.

### EXAMPLES

The experimental examples which follow were carried out using the materials and methods described hereinafter.

### Antibodies and Chemical Activators

Basiliximab (Simulect®, Novartis Pharma), cetuximab (Erbitux®, Merck Lipha sante), infliximab (Remicade®, Schering-plough), rituximab (Mabthéra®, Rituxan®, Roche) and abciximab (Réopro®, Lilly France) are chimeric therapeutic rMoAbs directed, respectively, against CD25, EGFR (epidermal growth factor receptor), TNFα, CD20 and the GPIIb/IIIa integrin. It should, however, be noted that abciximab does not contain an Fc portion. Alemtuzumab (Mabcampath®, Schering SA), bevacizumab (Avastin®, Roche), palivizumab (Synagis®, Abbott France) and trastuzumab (Herceptin®, Roche) are humanized rMoAbs directed, respectively, against CD52, VEGF (vascular endothelial growth factor), the respiratory syncytial virus fusion protein, and the ErbB2 antigen. Adalimumab (Humira®, Abbott France) is a completely human rMoAb directed against TNFα. Entanercept (Enbrel®, Wyeth-Lederle) is a TNFα receptor fused to an Fc portion of human IgG. The IVIgs (Tegeline®, LFB) are polyvalent human immunoglobulins prepared from human plasmas, and the anti-lymphocyte serum (Thymoglobuline®, Genzyme) is a rabbit serum directed against human thymocyte antigens. The anti-CD16 MoAb clone 3G8, the fluorescein isothiocyanate-conjugated anti-CD16 Ab (CD16-FITC) clone 3G8, the phycoerythrin-conjugated anti-IFNγ MoAb (IFNγ-PE) clone 45.15, the anti-CD56 MoAb conjugated to phycoerythrocyanin 5 (CD56-PC5) and to phycoerythrin (CD56-PE), and the -PE, -PC5 and -FITC isotypic controls all come from Beckman Coulter (Villepinte, France). They are all murine IgG1s. The phycoerythrocyanin-5-conjugated anti-CD107 MoAb (CD107-PC5) and the isotypic control-PC5 come from BD Biosciences (Le Pont de Claix, France).

The phorbol 12-myristate 13-acetate (PMA) and the calcium ionophore (CaI) A23187 were provided by Sigma (L′Isle d′Abeau Chesne, France).

### Culture of Target Cells

The target cell line used is the Daudi lymphoblastoid line. It is derived from a Burkitt's lymphoma. It is resistant to NK-mediated natural cytotoxicity (Dall'Ozzo et al., 2004) and expresses the human CD20 antigen at its surface. The Daudi cells were cultured in an incubator at 37° C. under 5% CO₂ in a humid atmosphere. The culture medium consisted of RPMI (Eurobio, Les Ulis, France) containing L-glutamine (Bio Whittaker Europe), 1 mM of sodium pyruvate (Invitrogen), 50 IU/ml of penicillin and 50 μg/ml of streptomycin (Bio Whittaker Europe). Twice a week, at the time the medium was changed, this mixture was supplemented with 10% of fetal calf serum (FCS) (Invitrogen) decomplemented at 56° C. for 30 min. The cells were cultured in flasks with a surface area of 75 cm² (BD Biosciences), in an average volume of 30 ml. The cell viability was regularly verified using the trypan blue exclusion test.

### Preparation of NK Cells

Peripheral venous blood samples (40 to 60 ml) were taken from healthy donors after written consent, in 6 ml tubes with an anticoagulant (sodium heparinate).

The blood was diluted to ⅔ in RPMI 1640 medium (Eurobio), and 7 ml was deposited onto 4 ml of Lymphoprep® (Eurobio) lymphocyte separation solution. After centrifugation for 20 min at 700 g, the mononuclear cells (PBMCs: peripheral blood mononuclear cells) formed a whitish ring at the Lymphoprep®/plasma interface, and this ring was recovered and then washed with 10 ml of RPMI 1640 medium. After centrifugation for 10 min at 560 g, the cell pellet obtained was taken up in 1 ml of PBS buffer containing 2 mM of EDTA (Sigma) and FCS at a final concentration of 10%: PES buffer (PBS/FCS/EDTA). Counting was performed, using a Mallassez hemocytometer, on a sample of 5 μl of cell suspension in 295 μl of trypan blue, under a microscope with a×40 objective.

The isolation of the NK cells from the PBMCs was carried out with the negative and indirect magnetic immunoselection kit (NK cell isolation kit II MACS®) from Miltenyi Biotec (Paris, France), according to the manufacturer's instructions: the PBMC suspension was centrifuged for 10 min at 560 g, and then taken up in PES buffer, at a rate of 40 μl per 10⁷ PBMCs. After the addition of 10 μl of solution A, containing a mixture of anti-CD3, anti-CD4, anti-CD14, anti-CD15, anti-CD19, anti-CD36, anti-CD123 and anti-CD235a (glycophorin A) MoAbs coupled to biotin, per 10⁷ PBMCs, incubation was carried out for 10 min at 4° C. 30 μl of PES buffer and 20 μl of solution B, containing beads coupled to an anti-biotin Ab, were then added per 10⁷ PBMCs. Incubation was then carried out for 15 min at 4° C., followed by washing in PES buffer and centrifugation for 10 min at 560 g. The pellet was taken up in 500 μl of PES buffer and loaded onto a magnetic separation column of LS VAR10MACS® type (Miltenyi Biotec) placed on a quadroMACS® magnet (Miltenyi Biotec). The NK cells were thus recovered in the eluted solution, and underwent washing with 5 ml of PES buffer. After centrifugation for 10 min at 560 g, these cells were taken up in 250 to 1000 μl of PES buffer, depending on the initial number of PBMCs. Counting using a Mallassez hemocytometer was carried out on a sample of 5 μl of cell suspension in 295 μl of trypan blue, under a microscope with a ×40 objective.

### Stimulation of NK Cells

Sensitization of culture plates with the therapeutic rMoAbs, the human IgGs, the anti-lymphocyte serum and the anti-CD16 MoAb

NUNC Maxisorp® 96-well plates (Fischer Labosi, Elancourt, France) were sensitized for 12 hours at 4° C. with concentrations of between 0.01 μg/ml and 10 μg/ml of each of the therapeutic rMoAbs or of etanercept or of human IgG1 or of human IgG3 or of polyvalent human IgG or of anti-lymphocyte serum or of the anti-CD16 MoAb 3G8, in a volume of 200 μl. The plate was then washed 3 times with PBS TWEEN (45 μl of TWEEN 20 (Sigma) in 100 ml of PBS), then saturated for 30 min with 1% bovine serum albumin (BSA) (Sigma), and again washed 3 times with PBS TWEEN before use. In the costimulation experiments, the plates were sensitized for 12 hours at 4° C. with 100 μl of a solution of anti-CD 16 MoAb clone 3G8 (Beckman Coulter) or of infliximab at 3 μg/ml, premixed or not premixed with 100 μl of a solution of MoAb at 10 or at 1 μg/ml: murine IgG1 clone MOPC-31C as control, anti-NKG2D MoAb clone 1D11, anti-CD11a MoAb clone G43-25B, anti-2B4 MoAb clone 2-69, anti-CD45 MoAb clone HI30 (from BD Biosciences), anti-CD2 MoAb clone 6F10.3, anti-NKp30 MoAb clone Z25, anti-CD56 MoAb clone C218 (from Beckman Coulter).

Stimulation of NK Cells with the Fixed Anti-CD16 MoAb

The NK cells were deposited in the sensitized plates at a rate of 10⁵ cells/well in a final volume of 200 μl, and incubated in the presence of 5 μl of anti-CD107-PECy5 or -PE MoAb and of a protein secretion blocker: BD GolgiPlug® containing brefeldin A (BD Biosciences®), at a rate of 1 μl/ml of cell solution, for 4 hours at 37° C., under 5% CO₂ in a humid atmosphere.

Stimulation of NK Cells with Daudi Cells in the Presence of Rituximab or of 3G8

The Daudi cells (2×10⁴) were deposited in flat-bottomed 96-well plates (Falcon 3047, BD Biosciences) with from 10⁴ to 2×10⁵ NK cells (effector-to-target ratio E:T of between 0.5:1 and 10:1) in the presence of 2 μg/ml of rituximab or of 3G8, 5 μl of the anti-CD107-PC5 MoAb and in the absence or presence of BD GolgiPlug®, at a rate of 1 μl/ml of cell solution, in a total volume of 200 μl, for 4 hours at 37° C., under 5% CO₂ in a humid atmosphere.

Stimulation of NK Cells with Daudi Cells in the Presence of Rituximab for Assaying the Secreted IFN7

The Daudi cells (2×10⁴) were deposited in flat-bottomed 96-well plates as described in the previous paragraph, in the presence of 2 μg/ml of rituximab in a total volume of 200 μl for 4 hours at 37° C., under 5% CO₂ in a humid atmosphere. 50 μl of supernatant was subsequently removed and then frozen at −80° C. with a view to the assay.

Stimulation of NK Cells with PMA and CaI

The NK cells were deposited in the plates at a rate of 10⁵ cells/well in a final volume of 200 μl, and incubated in the presence of 3.3 μg/ml to 100 μg/ml of PMA and/or of 33 μg/ml to 3300 μg/ml of CaI, in the presence of 5 μl of anti-CD107PECy5 MoAb and of BD GolgiPlug® at a rate of 1 μl/ml of cell solution, for 4 hours at 37° C., under 5% CO₂ in a humid atmosphere. For the kinetic study, the incubation times were between 1 h and 4 h.

### Detection of Intracellular IFN7

The detection of IFNγ was carried out according to the published techniques (Mendes et al., 2000; Vitale et al., 2000): after incubation, the NK cells were transferred into cytometry tubes (Beckman Coulter). The cells were washed with 2 ml of PBS buffer, centrifuged for 5 min at 560 g, and optionally incubated for 30 min at 4° C. with 5 μl of anti-CD16-FITC MoAb. The cells were then washed with 2 ml of PBS and again centrifuged for 5 min at 4° C. and incubated for 20 min at 4° C. with 270 μl of a BD cytofix/cytoperm Kit® solution (BD Biosciences). After washing with 2 ml of a Perm/Wash® washing solution (BD Biosciences), the cells were centrifuged for 5 min at 4° C., and then incubated for 30 min at 4° C. with 4 μl of anti-IFNγ-PE antibody. The cells were again washed with 2 ml of the Perm/Wash® washing solution, and centrifuged for 5 min at 4° C. at 560 g. Finally, the cell pellet was taken up in 400 μl of PBS buffer for the analysis of the cells by flow cytometry.

### Cytotoxicity Test

### Preparation of Target Cells

10⁷ Daudi cells were taken up in 300 μl of RPMI 1640 medium, and incubated with 3 MBq (100 μCi) of ⁵¹Cr₂O₄Na₂ (Dupont-NEN, Les Ulis, France) for 90 min at 37° C. with regular agitation. The cells were then washed twice with 10 ml of RPMI 1640 medium in order to remove the residual unbound radioactivity. After incubation for 60 min at 37° C., and two further washes in RPMI 1640 medium, the cell suspension was adjusted to a concentration of 4×10⁵ cells/ml. The cell suspension was then distributed into a flat-bottomed, 96-well Microtest® plate (BD Biosciences), at a rate of 50 μl (2×10⁴ cells) per well.

### Measurement of Cell Cytotoxicity

50 μl of rituximab at a final concentration of 2 μg/ml were added to 2×10^{4 51}Cr-labeled Daudi cells. The effector cells were then distributed, in 100 μl, into the wells of interest at E:T ratios of between 0.5:1 and 10:1. After incubation for 4 h at 37° C. under CO₂, 50 μl of supernatant were recovered and evaporated overnight in a dry incubator. The radioactivity was measured using a γ-particle counter (Top Count®, Packard Rungis France). The spontaneous release (cpm spont) was obtained by incubating target cells with 150 μl of culture medium alone, and the maximum release (cpm max) was obtained by incubating target cells with 150 μl of culture medium containing 1% of Triton X100 (Sigma). The % specific lysis was given by the calculation:

% specific lysis=(cpm test−cpm spont)/(cpm max−cpm spont).

### Assaying of Secreted IFN7

The assaying of the IFNγ was carried out on the supernatants with an enzyme immunoassay kit for IFNγ (Enzyme Immunoassay Kit®, Immunotech), according to the procedure provided. It is a two-stage sandwich enzyme immunoassay: capture of the IFNγ by an anti-IFNγ monoclonal antibody fixed in the wells of a microtitration plate, then binding of a biotinylated anti-IFNγ second Ab, revealed by a streptavidin-peroxidase conjugate. The enzyme activity was then determined by adding a chromogenic substrate. The intensity of coloration developed, which is read at 450 nm, is proportional to the concentration of IFNγ present in the sample or the standard. The results are expressed in IU/ml.

Detection of the Fixed rMoAbs or of the Fixed Anti-CD16 MoAb

The NUNC plates sensitized with the rMoAbs were washed, and then a dilution to 1/4000th of γ-chain-specific goat anti-human IgG F(ab′)₂ conjugated to peroxidase (Sigma) was deposited at a rate of 100 μl per well. The NUNC plates sensitized with the anti-CD16 MoAb 3G8 were washed, and then a dilution to 1/5000th of peroxidase-conjugated goat antibody specific for the mouse IgG Fc portion (Jackson Immuno Research Laboratories, Villepinte France) were deposited at a rate of 100 μl/well. After incubation for 1 h at ambient temperature, followed by three washes with a solution of PBS-TWEEN (45 μl of TWEEN 20 (Sigma) in 100 ml of PBS), a solution of O-phenylenediamine (Sigma) at a final concentration of 0.5 mg/ml was added, at a rate of 100 μl per well. After incubation for 30 min at ambient temperature, 50 μl of a 2N solution of H₂SO₄ (STOP solution) was deposited in each well. The plate was read on an iEMS reader MF® spectrophotometer (Labsystems, Cergy pontoise France) at a wavelength of 492 and 620 nm. The absorbance values obtained corresponded to the difference in the absorbances obtained between 492 and 620 nm.

Measurement of the Affinity of the rMoAbs and of the Human IgGs for the FcγRIIIa of NK Cells

The purified NK cells were adjusted to the concentration of 15×10⁶ cells/ml in PBS. 10 μl of this solution were incubated with 90 μl of the indicated concentrations of the therapeutic rMoAbs or of entanercept or of polyvalent human IgGs in PBS in 5 ml cytometer tubes (Beckman Coulter) for 30 min at 4° C. The cells were then incubated with 5 μl of anti-CD56-PC5 MoAb and 45 μl of anti-CD16-FITC MoAb 3G8 at 0.334 μg/ml (i.e. a final concentration of 0.1 μg/ml) for 30 min at 4° C. After washing with 2 ml of PBS, the cells were taken up in 450 μl of PBS and analyzed by flow cytometry. The results were expressed as % inhibition of the binding of the anti-CD16 MoAb 3G8 according to the following equation:

%=(Xmean in the absence of MoAb−Xmean in the presence of rMoAb)×100/(Xmean in the absence of rMoAb).

Binding of C1q to the Therapeutic rMoAbs and to the Human IgGs Adsorbed

The plates sensitized with 5 μg/ml of the various rMoAbs or of human IgG1 or of human IgG3 or of human polyvalent IgGs or of the anti-CD16 MoAb 3G8 were washed and then incubated for 2 h at ambient temperature with the indicated concentrations of human C1q (Sigma) diluted in PBS. After 3 washes with a solution of PBS-TWEEN, the plates were incubated with a peroxidase-coupled sheep anti-human C1q polyclonal antibody (Biogenesis, distributed by Absys, Paris, France), diluted in PBS-1% BSA. After incubation for 1 h at ambient temperature, followed by three washes with a solution of PBS-TWEEN, a solution of O-phenylenediamine at a final concentration of 0.5 mg/ml was added, at a rate of 100 μl per well. After incubation for 15 min at ambient temperature, 50 μl of a 2N solution of H₂SO₄ were deposited in each well. The plate was read on an iEMS reader MF® spectrophotometer at a wavelength of 492 and 620 nm. The absorbance values obtained corresponded to the difference in the absorbances obtained between 492 and 620 nm.

### Analysis by Flow Cytometry

The cells were analyzed with an XL cytometer (Beckman Coulter) using the EXPO 32® acquisition and analysis software. For each determination, a minimum of 4000 cells were analyzed. The analysis of the fluorescences was conditioned by a window on a bi-parametric size/structure diagram (FSC/SSC=forward scatter channel/side scatter channel).

### Example 1

### Measurement of the ADCC Exerted by NK Cells on Daudi Cells in the Presence of Rituximab Using the ⁵¹Cr-Release Test and Using the Membrane Expression of CD107

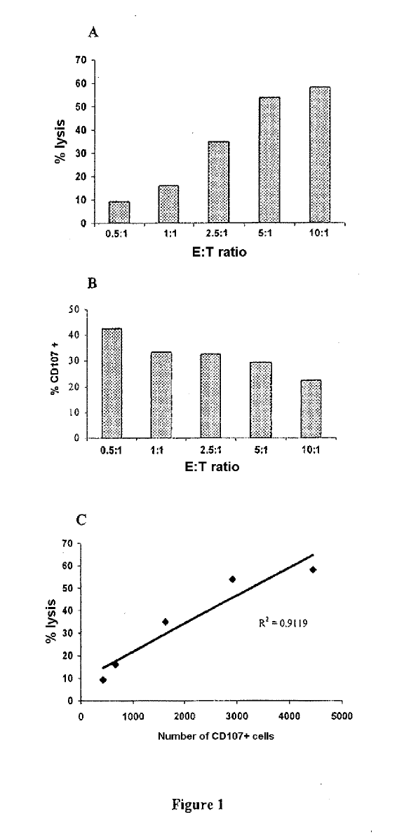

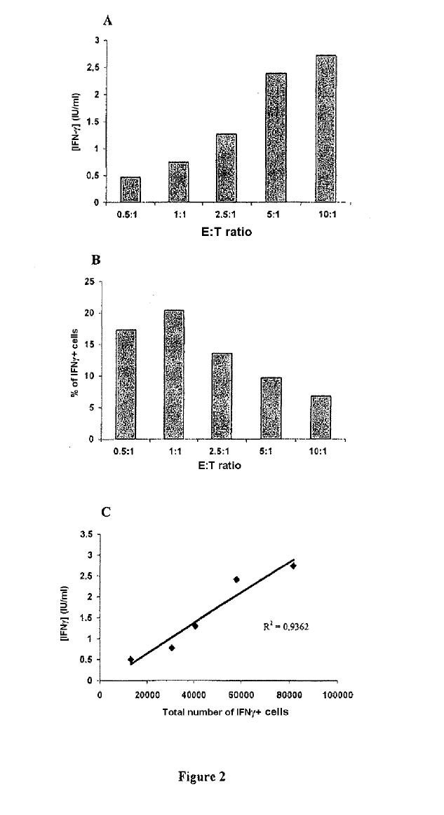

It has previously been shown that purified NK cells effectively lyse CD20+Daudi target cells in the presence of rituximab (anti-CD20 chimeric antibody) at the concentration of 2 μg/ml in a ⁵¹Cr-cytotoxicity test for 4 h (Dall'Ozzo et al., 2004). It has recently been shown that the expression of CD107 on cytotoxic cells makes it possible to evaluate the degranulation of said cells and represents an alternative measurement to cytotoxicity (Alter et al., 2004; Betts et al., 2003; Betts et al., 2004). In order to test the validity of the degranulation test for evaluating the ADCC, from 10⁴ to 2×10⁵ purified NK cells from the same donor were incubated for 4 h at 37° C. in the presence of 2 μg/ml of rituximab, with 2×10⁴ Daudi cells prelabeled or not prelabeled with ⁵¹Cr. The cytotoxicity was measured by counting the radioactivity released into the supernatant by the Daudi cells preincubated with the ⁵¹Cr. The degranulation was evaluated by flow cytometry, after incubation of the effectors and of the nonlabeled targets during the 4 h of the test with an anti-CD107 MoAb conjugated to PC5, and then labeling with an anti-CD 56 MoAb conjugated to PE. As shown by FIG. 1A and as expected, the percentage lysis of the Daudi cells increased with the E:T ratio.

FIG. 1B shows that only a fraction (in all cases <50%) of the CD56+NK cells expressed CD107 after the 4 h of incubation. Furthermore, contrary to the lysis percentages presented in FIG. 1A, the percentages of CD56+CD107+ cells decreased with the increase in the E:T ratio. However, the absolute number of CD107+NK cells calculated from the number of NK cells initially added for each E:T ratio, is well correlated with the percentage lysis of the Daudi cells for a preparation of NK cells originating from a given donor.

### Example 2

### Measurement of the ADCP Exercised by NK Cells with Respect to Daudi Cells in the Presence of Rituximab by Assaying of the Secreted IFNγ and by Detection of the Producer Cells

In order to study whether ADCP and ADCC can be exercised simultaneously by NK cells, the IFNγ production by NK cells was evaluated under the previous experimental conditions: 10⁴ to 2×10⁵ purified NK cells from the same donor were incubated for 4 h at 37° C. in the presence of 2 μg/ml of rituximab, with 2×10⁴ unlabeled Daudi cells. The IFNγ was assayed in the supernatant by means of an ELISA technique. As shown by FIG. 2A and as was the case for the lysis percentages, the IFNγ concentration in the supernatant increased with the E:T ratio. In order to determine whether all the cells synthesize IFNγ, the NK cells originating from the same donor were incubated under identical experimental conditions, but in the presence of brefeldin A, which blocks cell transport mechanisms, and therefore secretion. The cells were subsequently labeled with an anti-CD56 MoAb conjugated to PC5, permeabilized and incubated with an anti-IFNγ MoAb conjugated to PE, and the percentage of NK cells expressing intracellular IFNγ was evaluated by flow cytometry according to the published techniques (Mendes et al., 2000; Vitale et al., 2000). In the absence of brefeldin A, no CD56+IFNγ+ cell was detected (results not shown). On the other hand, FIG. 2B shows that a fraction of the CD56+ cells was IFNγ+ after the 4 h of incubation in the presence of brefeldin A. However, this fraction was relatively small (in all cases <25%). In addition, unlike the IFNγ concentrations (FIG. 2A), the percentages of CD56+IFNγ+ cells decreased when the E:T ratio increased between 1.25:1 and 10:1. However, the absolute number of IFNγ+NK cells calculated from the number of NK cells initially added for each E:T ratio, in the presence of brefeldin A, is well correlated with the concentration of IFNγ produced by the NK cells originating from the same donor in the absence of brefeldin A.

### Example 3

### Simultaneous Study of the Degranulation and of the Synthesis of IFNγ by the NK Cells Stimulated with Daudi Cells in the Presence of Rituximab or of 3G8

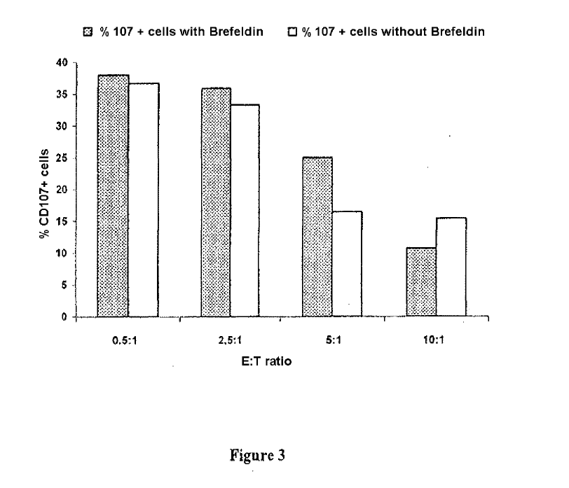

The results of FIGS. 1B and 2B show that, under the experimental conditions used, neither ADCC nor ADCP is exerted by all the NK cells. In order to study whether the two responses are exerted at least in part by the same effector cells, it was necessary to simultaneously study the expression of CD107 and of intracellular IFNγ, which could only be done in the presence of brefeldin A. In order to study the possible influence of brefeldin on the expression of CD107, purified NK cells were incubated under the conditions of FIG. 1A with Daudi cells in the presence of 2 μg/ml of rituximab, in the presence of the anti-CD107 MoAb and in the presence or absence of brefeldin, and then labeled with the anti-CD56 MoAb and analyzed by flow cytometry. As shown by FIG. 3, the percentages of CD56+CD107+ cells obtained in the absence or in the presence of brefeldin A was similar (white histograms versus histograms with dots).

The above result therefore made it possible to simultaneously study the degranulation and the synthesis of IFNγ by NK cells. For this, purified NK cells were incubated for 4 h at 37° C. with Daudi cells in the presence of brefeldin A, and of the anti-CD107 MoAb, and in the absence or presence of 2 μg/ml of rituximab or of the murine anti-CD16 MoAb 3G8. The latter is used in order to carry out a redirected stimulation test. Specifically, Daudi cells express the CD32/FcγRII molecule which has affinity for the Fc portion of murine MoAbs of IgG1 isotype, such as 3G8. Under these conditions, the MoAb 3G8 bound to the target can recruit, via its recognition site, NK cells which express CD16/FcγRIIIa. This binding of the MoAb to FcγRIIIa/CD16 activates the effector cell and induces its response with respect to the target. The high affinity of 3G8 for FcγRIIIa/CD16 allows optimal stimulation (Dall'Ozzo et al., 2004). The cells were subsequently labeled with the anti-CD56 MoAb, permeabilized, and incubated with the anti-IFNγ MoAb and then analyzed by flow cytometry.

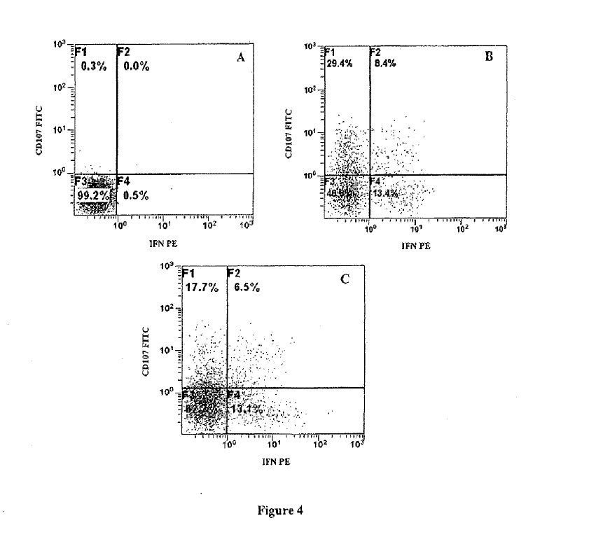

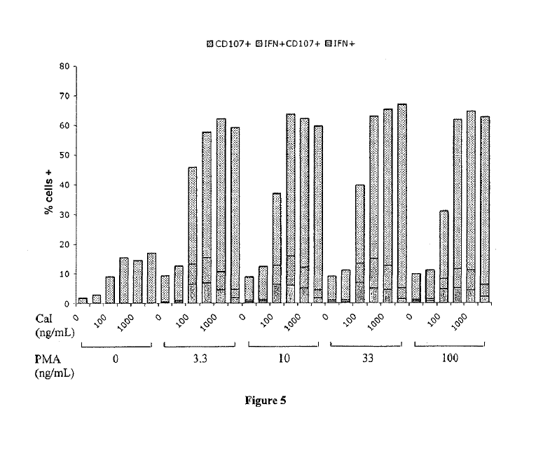

The cytograms of FIG. 4 show that no CD107+ or IFNγ+NK cell was detected in the absence of stimulant (cytogram A). Conversely, significant percentages of CD107+NK cells and of IFNγ+NK cells are observed after stimulation in the present of rituximab or of 3G8 (cytogram B and C). While a fraction of the NK cells is both CD107+ and IFNγ+ (upper right quadrant), showing that ADCC and ADCP are not mutually exclusive, the majority of the responder cells are exclusively CD107+ (upper left quadrant) or exclusively IFNγ+ (lower right quadrant). The results obtained with rituximab and 3G8 were comparable. It should be noted that, under these experimental conditions, the responder cells represented only a minority fraction of the NK cells.

### Example 4

### Simultaneous Study of the Degranulation and of the Synthesis of IFNγ by the NK Cells Stimulated with PMA and CaI

The above results showed that, in response to FcγRIIIa being engaged by rituximab or 3G8, the NK cells which degranulate and those which synthesize IFNγ belong to populations which are at least partly different. In order to study whether this functional dichotomy of NK cells could be observed under CD16-independent stimulation conditions, purified NK cells were stimulated with various concentrations of the calcium ionophore (CaI) A23187 and of phorbol myristate acetate (PMA) used alone or in combination. This is because it has been reported that stimulation with this combination enables, on the one hand, degranulation of NK cells (Atkinson et al., 1990) and, on the other hand, synthesis of cytokines (Mendes et al., 2000). The activation was carried out in a culture plate, wherein the NK cells were incubated for 4 h at 37° C. under CO₂, in the presence of the anti-CD107 MoAb, and then the cells were permeabilized, labeled with the anti-IFNγ MoAb and analyzed by flow cytometry.

No IFNγ+ cell was detected after stimulation with CaI in the absence of PMA or with PMA in the absence of CaI (FIG. 5). Conversely, stimulation of the cells with concentrations of CaI above 100 ng/ml induced the expression of CD107 on the NKs. The expression was at a maximum (around 15% of CD107+ cells) from the concentration of 330 ng/ml of CaI onward. Similarly, the stimulation with concentrations of 3.3 to 100 ng/ml of PMA induced the expression of CD107 on approximately 10% of the NK cells. When the NK cells were stimulated with the combination of the two stimulants, the expression profile of CD107, like that of IFNγ, was identical for all the concentrations of PMA used (from 3.3 to 100 ng/ml). Thus, the effect of the Cal was synergic with that of the PMA for CaI concentrations above 100 ng/ml, both in terms of the expression of CD107 and in terms of the synthesis of IFNγ, and the two responses were at a maximum at the concentration of 330 ng/ml. On the other hand, the expression of CD107 remained at a maximum for concentrations of 1000 to 3300 ng/ml, although the synthesis of IFNγ substantially decreased at these same concentrations. Finally, as previously, three populations of responder cells were observed under all the stimulation conditions: CD107+ cells, IFNγ+ cells and CD107+IFNγ+ cells. The latter represented only a minority fraction of all the responder cells, i.e. from 10 to 20% of the CD107+ cells, but generally more than 50% of the INFγ+ cells.

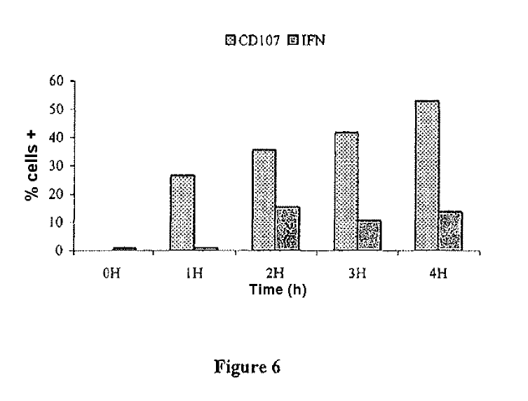

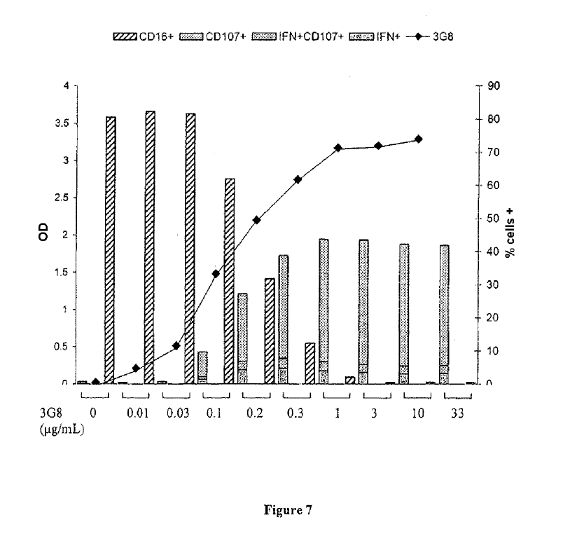

The expression kinetics for CD107 and for intracellular IFNγ were also studied after stimulation of the cells with a combination of 330 ng/ml of CaI and 33 ng/ml of PMA. The purified NK cells were stimulated in the presence of brefeldin A and of anti-CD107 MoAb. After incubation times of between 0 and 4 h, the cells were permeabilized and then labeled with the anti-IFNγ MoAb and analyzed by flow cytometry. FIG. 6 shows that the expression of CD107 on the NK cells was already considerable after the first hour of stimulation and that the percentages subsequently increased evenly over time. The IFNγ+NK cells were detected after 2 h of stimulation and the percentages obtained did not increase after longer stimulation times.

### Example 5

### Simultaneous Study of the Loss of CD16, of Degranulation and of the Synthesis of IFNγ by NK Cells Stimulated with an Anti-CD16 Antibody Fixed on a Plastic Support

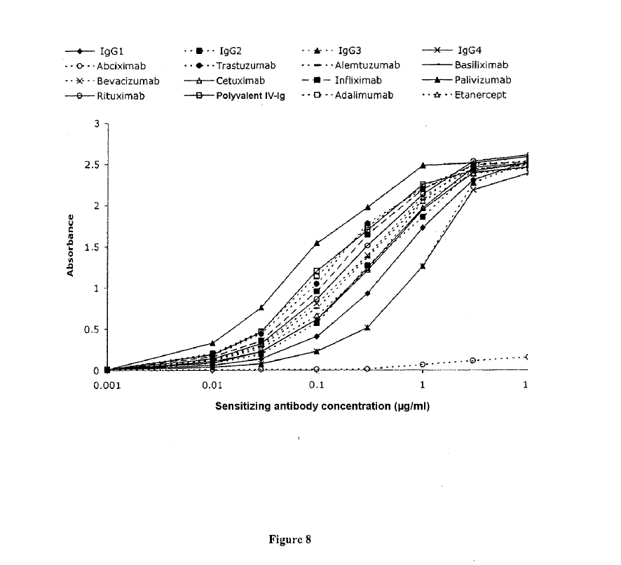

In order to study the effect of the engagement of only FcγRIIIa on the responses of NK cells, purified NK cells were incubated for 4 h at 37° C. in the presence of brefeldin A and of the anti-CD107 MoAb, in wells of a culture plate onto which varying amounts of the anti-CD 16 MoAb 3G8 had previously been adsorbed. The amounts of 3G8 effectively fixed at the bottom of the wells of the culture plate were evaluated by an ELISA technique using an anti-mouse IgG antibody labeled with an enzyme. The results are given as absorbance values (FIG. 7: left-hand y-axis) relative to the concentration of 3G8 used during the adsorption (FIG. 7: x-axis). The cells were subsequently labeled with an FITC-conjugated anti-CD16 MoAb. This is because it has been reported that the bridging of CD16 with an anti-CD16 MoAb is reflected by loss of membrane expression of the molecule on NK cells (Borrego et al., 1994). The cells were finally permeabilized, incubated with the anti-IFNγ MoAb and analyzed by flow cytometry. The results were expressed as percentage of positive cells (FIG. 7: right-hand y-axis).

FIG. 7 shows that saturation of the wells of the plate with the MoAb 3G8 was obtained for a concentration of the order of 1 μg/ml. After incubation of the NK cells in the wells sensitized with the various concentrations of the MoAb 3G8, a decrease in membrane expression of CD16 was observed starting from 0.1 μg/ml of 3G8, which represents the concentration resulting in approximately 50% saturation. The membrane expression of CD16 then very greatly decreased as the concentration increased (approximately 15% of positive cells at 0.3 μg/ml corresponding to approximately 85% saturation) and became undetectable at the saturating concentrations. Thus, close to 100% of the NK cells lost the expression of CD16 and were therefore activated, after incubation in the wells sensitized with a saturating concentration of 3G8. CD107+ and/or IFNγ+NK cells were detected after incubation in wells sensitized with a concentration of 0.1 μg/ml. The percentages of CD107+ and/or IFNγ+NK cells then increased as the concentration of 3G8 increased, and reached a plateau for a concentration of 0.3 μg/ml. The three populations of responder cells were again observed under these stimulation conditions.

### Example 6

### Comparative Study of the Sensitization of the Plates with Several Therapeutic rMoAbs and with Human IgGs

In order to be able to compare the functional responses induced by the various rMoAbs adsorbed onto a plastic support, it was necessary to compare, beforehand, their adsorption onto said support. The wells of the microplates were therefore sensitized overnight at 4° C. with concentrations of between 0.01 and 10 μg/ml of rMoAb or of human IgG of each of the four subclasses or of polyvalent human IgG or of etanercept (fusion protein comprising an Fc portion). The binding was revealed by ELISA using a peroxidase-coupled anti-human Fc goat Ab. FIG. 8 shows, for each of the products tested, the absorbance profile and therefore binding profile as a function of the sensitizing concentration. As expected, the binding of abciximab, which does not have an Fc portion, was not detected in this experimental system. On the other hand, for all the rMoAbs or IgGs tested, the binding increased as the sensitizing concentration increased, to a plateau, according to a sigmoid profile. The curves obtained with the IgG3, the IgG4 and, to a lesser extent, with the IgG1 were shifted to the right, indicating slightly less sensitization with these myelomatous proteins than with the rMoAbs. Conversely, the curve obtained with palivizumab was slightly shifted to the left, showing better sensitization of the plate with this rMoAb. The results obtained with the other products were similar. For all the products tested, with the exception of palivizumab, saturation of the plate was obtained with sensitizing concentrations of between 1 and 3 μg/ml.

### Example 7

### Comparative Study of the Binding of C1q by Several Therapeutic rMoAbs and by Human IgGs

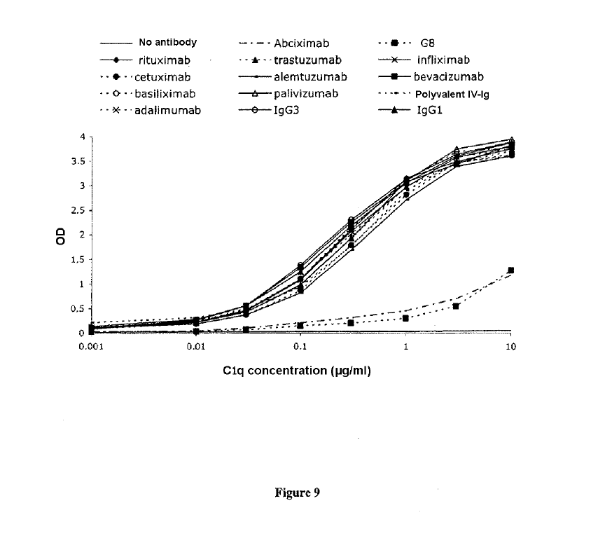

In order to confirm that the binding of the various rMoAbs or human IgGs was equivalent at the saturating sensitizing concentrations, the plates were sensitized with 5 μg/ml of each of the rMoAbs or of human IgG, and then incubated with concentrations of between 0.01 and 10 μg/ml of human C1q, which binds to the CH2 domain of the Fc portion. The binding of C1q was revealed by ELISA using a peroxidase-coupled anti-human C1q sheep Ab. FIG. 9 shows, for each of the products tested, the absorbance profile, and therefore the profile for binding of C1q, as a function of the concentration of C1q. As expected, C1q did not bind to abciximab, which does not have an Fc portion, nor to 3G8, which is a murine MoAb not recognized by human C1q. On the other hand, considerable binding was observed with all the other rMoAbs or IgGs tested. This binding increased as the concentration increased, to a plateau, according to a sigmoid profile. All the profiles obtained were identical, demonstrating that, whatever the rMoAb or human IgG used, the sensitizing concentration of 5 μg/ml allows effective saturation of the plate. These results also show that no variability exists between the rMoAbs tested for the binding of C1q, and that said binding does not require the Ab to be bound to the Ag for which it is specific.

### Example 8

### Comparative Study of the Affinity of Several Therapeutic rMoAbs and of Human IgGs for the FcγRIIIa of NK Cells

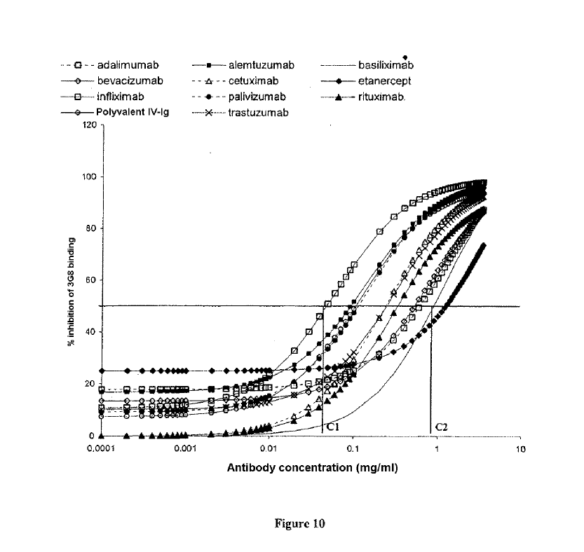

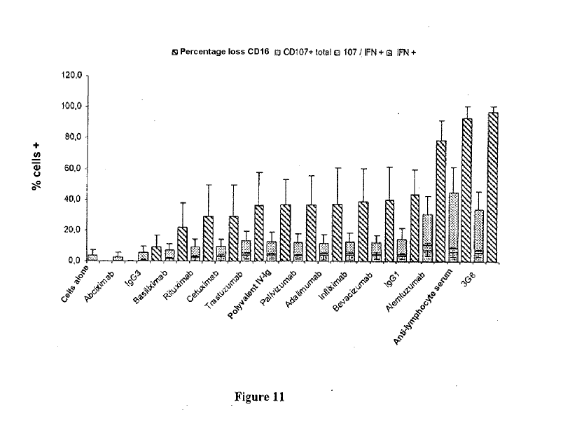

The functional response of cells expressing FcγRIIIa with respect to a target sensitized with an rMoAb depends on the affinity of the receptor for the rMoAb (Dall'Ozzo et al., 2004). In order to compare the functional response induced by various rMoAbs adsorbed onto the plate, it was necessary to compare their affinity for the FcγRIIIa expressed on the responder cells. For this, the ability of concentrations of between 0.001 and 10 mg/ml of each of the rMoAbs or of polyvalent IgG to inhibit the binding of the FITC-conjugated anti-CD16 MoAb 3G8 to the purified NK cells of a donor was studied by flow cytometry as described previously (Dall'Ozzo et al., 2004). FIG. 10 shows, for each of the products tested, the inhibition of the binding of 3G8 as a function of the concentration of the product tested. For all the rMoAbs tested and for the polyvalent IgG, the inhibition of the binding of 3G8 increased as the concentration increased, to a plateau, according to a sigmoid profile. However, the inhibition percentages observed depended greatly on the product tested. Thus, the concentration C1 of infliximab making it possible to inhibit 50% of the binding of 3G8 is approximately 20 times lower than the concentration C2 of basiliximab producing the same effect, reflecting the better affinity of the first for the FcγRIIIa expressed on the NK cells. In total, although the commercially available therapeutic rMoAbs are all IgG1s and they therefore possess an Fc portion having a conserved sequence, they exhibit varying affinities for FcγRIIIa.

### Example 9

### Comparative Study of the Loss of CD16, of Degranulation and of IFNγ Synthesis by NK Cells Incubated in Sensitized Plates, as a Function of the rMoAbs or of the IgGs Sensitizing the Plastic Support

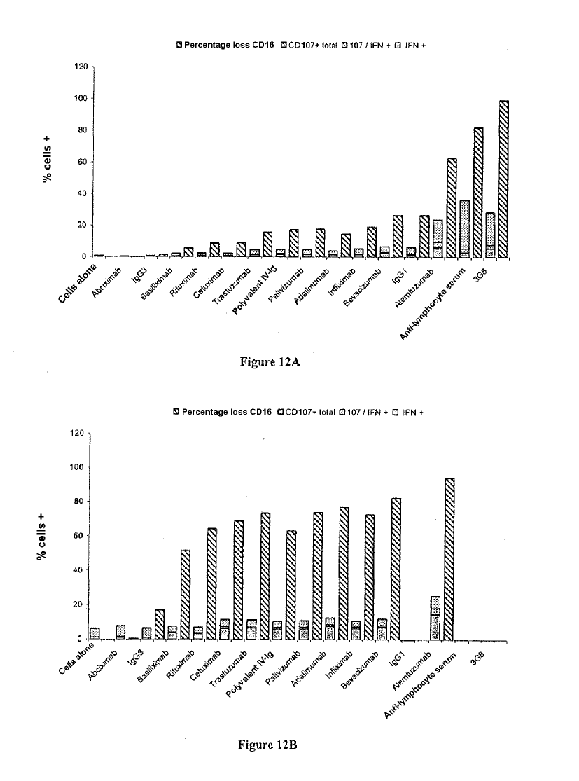

In order to compare the responses of NK cells to the various rMoAbs, to human IgGs, to the anti-lymphocyte serum and to the anti-CD16 MoAb 3G8, the purified NK cells were incubated for 4 h at 37° C. in the presence of brefeldin A and of the anti-CD107 MoAb, in wells of a culture plate presensitized or not presensitized with a saturating concentration 3 μg/ml) of each of the rMoAbs or of polyvalent IgG or of IgG1 or of IgG3 or of anti-lymphocyte serum or of the anti-CD16 MoAb 3G8. The cells were then labeled with an anti-CD16 MoAb. The cells were finally permeabilized, incubated with the anti-IFNγ MoAb and analyzed by flow cytometry. FIG. 11 shows for each of the products tested, on the one hand, the mean percentage of NK cells having experienced a loss of membrane expression of CD16 and, on the other hand, the mean percentages of CD107+ and/or IFNγ+ cells (results classed in increasing order of loss of CD16 and obtained over the course of 8 different experiments). As was previously shown in FIG. 7, the NK cells incubated in nonsensitized wells did not detectably lose CD16 and did not express CD107 or intracellular IFNγ. The same was true for the NK cells incubated in wells sensitized with abciximab. For all the other products, both responses were detected. For each product tested, the percentage of cells having had the functional response (sum of the percentages of CD107+, CD107+IFNγ+ and IFNγ+ cells) was always less than the percentage of activated cells (percentage of cells having lost CD16). However, for all the products tested, the percentages of cells having had a functional response was very well correlated with the percentages of activated cells. As was previously shown in FIG. 7, close to 100% of the NK cells incubated in wells sensitized with the anti-CD16 MoAb 3G8 lost the expression of CD16 and were therefore activated. Approximately ⅓ of the NK cells also had a functional response, among which a majority (close to 85%) expressed CD107 alone or associated with intracellular IFNγ. Similar results were observed with the NK cells incubated in wells sensitized with the anti-lymphocyte serum. The NK cells incubated in wells sensitized with human IgG3 experienced a very slight loss of CD16 and had a very weak functional response. The responses (loss of CD16 and functional responses determined by CD107 and intracellular IFNγ) of the NK cells incubated in wells sensitized with rMoAbs varied substantially, according to the MoAb used. Basiliximab induced the weakest responses (on average, slightly more than 20% of cells having lost CD16), then cetuximab and rituximab (on average, slightly less than 30% of cells having lost CD16), then trastuzumab, palivizumab, adalimumab, infliximab and bevacizumab brought about stronger responses close to those obtained with the polyvalent IgG and the human IgG1 (on average, between 36.5% and 43.4% of cells having lost CD16). Finally, alemtuzumab induced a massive response close to that observed with 3G8 (on average, 78.5% of cells having lost CD16). Among the NK cells having had a functional response, a majority expressed CD107 alone or associated with IFNγ. However, the proportion of CD107+ cells was lower (between 58% and 72%) than that observed after incubation of the cells in wells sensitized with the anti-CD16 3G8 or the anti-lymphocyte serum. Overall, the sensitization of the plates with a saturating concentration of various rMoAbs led to variable functional responses and activation on the part of the NK cells, as a function of the rMoAbs used. These results therefore show that, independently of the antigens that they recognize, the various rMoAbs activate NK cells differently, via their Fc portion, and that the method used makes it possible to measure and to compare the responses produced by these various rMoAbs.

### Example 10

### Comparative Study of the Loss of CD16, of Degranulation and of IFNγ Synthesis by NK Cells Incubated in Sensitized Plates, as a Function of the Cell Donor

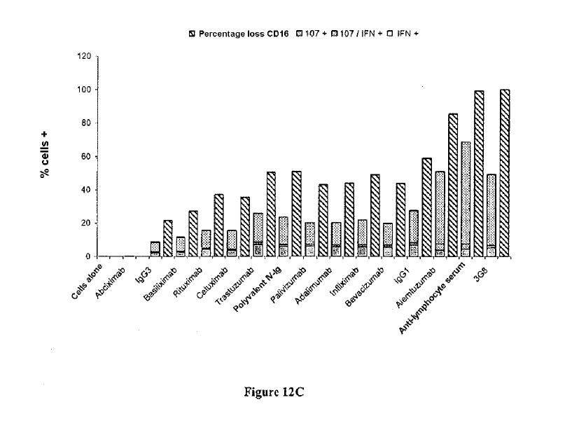

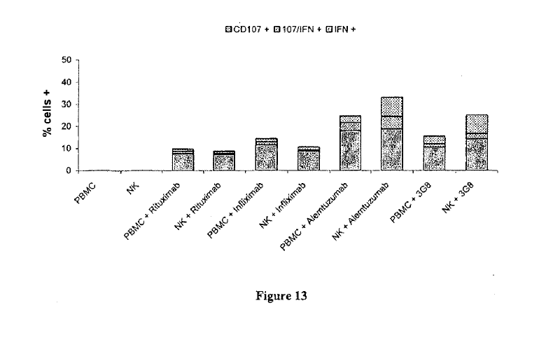

The individual results for the activation and for the functional response of three among the eight experiments of FIG. 11 are given in FIG. 12. In accordance with the results shown in FIG. 11, the NK cells of the three donors (FIGS. 12A, 12B and 12C) had zero or very weak responses after incubation in nonsensitized wells or wells sensitized with abciximab, and showed very strong responses after incubation in the wells sensitized with the anti-CD16 MoAb 3G8, the anti-lymphocyte serum and alemtuzumab. The responses to the other products tested varied greatly with the donor. Thus, the NK cells of the first donor (FIG. 12A) experienced a loss of CD16 which was much less than the mean (from 1% for IgG3 to 27% for IgG1) and had functional responses that were also very weak. The NK cells of the second donor experienced a loss of C16 which was much higher than the mean (from 17% for IgG3 to 82% for IgG1) and had intermediate functional responses. In addition, among the NK cells of this donor which had had a functional response, a minority expressed CD107 alone or associated with IFNγ, including after stimulation with alemtuzumab. Finally, the NK cells of the third donor experienced a loss of CD16 (from 22% for IgG3 to 57% or IgG1) and had functional responses slightly greater than the mean. Among the NK cells of this donor which had had a functional response, a large majority expressed CD107 alone or associated with IFNγ, whatever the product tested. Overall, the activation and the functional responses of the NK cells, after incubation in plates sensitized with a saturating concentration of certain rMoAbs or of human IgG, vary qualitatively and quantitatively as a function of the cell donor. These results therefore show that the responses to the Fc portion of certain rMoAbs or human IgGs depend on the origin of the NK cells, and that the method used makes it possible to measure and to compare the responses produced by the NK cells of the various donors.

### Example 11

### Comparative Study of Degranulation and of IFNγ Synthesis by NK Cells and by PBMCs from the Same Donor, Incubated in Sensitized Plates

In order to verify that the method can be used without an NK-cell purification step, the PBMCs and the NK cells from the same donor were prepared and incubated for 4 h at 37° C. in the presence of brefeldin A and of the anti-CD107 MrAb, in wells of a culture plate presensitized or not presensitized with a saturating concentration of rituximab, of infliximab, of alemtuzumab or of the anti-CD16 MoAb 3G8. The cells were subsequently labeled with an anti-CD56 MoAb. The cells were finally permeabilized, incubated with the anti-IFNγ MoAb and analyzed by flow cytometry. Figure shows, for each of the products tested, the percentages of CD107+ and/or IFNγ+ cells among the CD56+NK cells and among the CD56+PBMCs. The percentages of responder cells obtained with the purified NK cells and with the CD56+PBMCs were comparable.

### Example 12

### Study of the Loss of CD16, of Degranulation and of IFNγ Synthesis by NK Cells Incubated in Plates Cosensitized with 3G8 or Infliximab and with MoAbs Directed Against NK Cell Activation or Coactivation Molecules

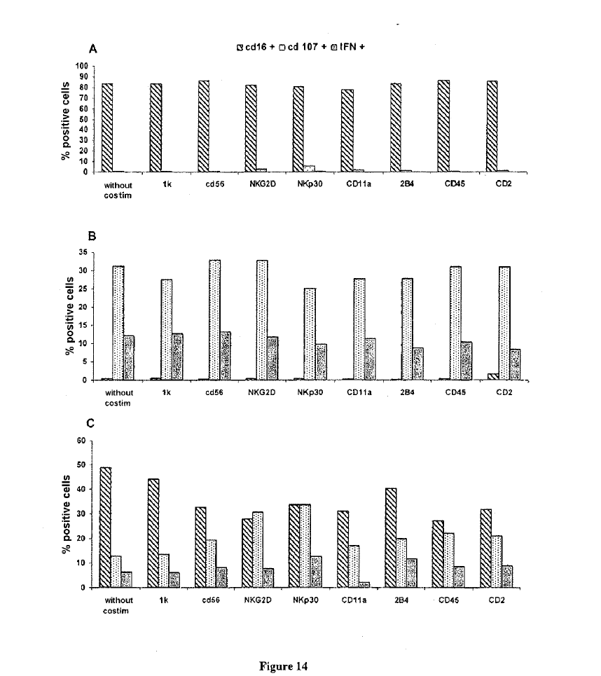

It has been shown that the receptors NKp30, NKp46 and NKG2D are involved in natural cytotoxicity (Pende et al., 2001), and that receptors such as CD2, 2B4 (CD244) or LFA-1 (CD11a/CD18) are involved in the coactivation of the NK cell (Bryceson et al., 2006; Natarajan et al., 2002). In addition, NKG2D appears to have a role in natural cytokine synthesis by NK cells (Andre et al., 2004). Similarly, the membrane molecule CD45 appears to be essential for the production of cytokines by mouse NK cells, but not for the cytotoxicity exerted by these same cells (Hesslein et al., 2006; Huntingon et al., 2005). In order to study the effect of the stimulation of these molecules simultaneously with the engagement of FcγRIIIa, on the functional orientation of NK cells, plates sensitized with antibodies directed against these molecules or against the CD56 adhesion molecule (which does not have any coactivating activity) or against a murine IgG1, and cosensitized or not cosensitized with the anti-CD16 antibody 3G8 or with infliximab (1.5 μg/ml), were used. The stimulation and the labelings were carried out as described in example 9. FIG. 14A shows the percentages of CD16+, CD107+ and IFNγ+NK cells of a donor after incubation in nonsensitized plates or plates sensitized only with the control IgG1 or with the MoAbs directed against the CD56, NKG2D, NKP30, CD11a, CD244, CD45 or CD2 molecules. The NK cells incubated under these conditions did not detectably lose CD16 and expressed neither CD107 (with the exception of a very weak expression after incubation in the presence of the anti-NKP30 MoAb) nor intracellular IFNγ. Conversely, virtually all the NK cells incubated in plates sensitized with the MoAb 3G8 (FIG. 14B) experienced a loss of CD16 expression, whether or not the plates had been cosensitized with the various MoAbs directed against the activation molecules tested. In accordance with this result and with those given in FIGS. 11 and 12, a significant fraction of these cells were CD107+(from 25 to 33%) or IFNγ+(from 9 to 13%). However, the responses were not substantially modified by the coengagement of the activation or coactivation molecules tested. Finally, as shown by FIG. 14C, a significant fraction of the NK cells of this donor, incubated in plates sensitized with infliximab, experienced a loss of CD16 expression (from 47% of the cells incubated in the wells sensitized with infliximab and the control murine IgG1 to 68% of the cells incubated in wells cosensitized with infliximab and the anti-CD45 MoAb). In accordance with this result and with those given in FIGS. 11 and 12, a significant fraction of these cells were CD107+ or IFNγ+, but the percentages varied substantially as a function of the activation or coactivation molecules tested. Thus, 13.6% of the NK cells were CD107+ after incubation in wells cosensitized with the murine IgG1, against approximately 20% of the cells incubated in wells cosensitized with the MoAbs directed against CD56, CD11a, CD244, CD45 and CD2, and approximately 30% of the cells incubated in wells cosensitized with the MoAbs directed against NKG2D and NKP30. Similarly, from 6.1 to 9% of the NK cells were IFNγ+ after incubation in wells cosensitized with the murine IgG1, or with the MoAbs directed against CD56, NKG2D, CD45 and CD2, against only 2% of the cells incubated in wells cosensitized with the anti-CD11a MoAb and against 12% to 13% of the cells incubated in wells cosensitized with the anti-NKP30 and anti-CD244 MoAbs. Thus, the co-engagement of the NKP30 molecule increased the two functional responses induced by infliximab, that of NKG2D increased only degranulation, that of CD244 increased only IFNγ synthesis, whereas that of CD11a decreased the latter. Overall, these results therefore show that, under our experimental conditions, engagement of only the activation or coactivation molecules of NK cells did not induce a detectable response. On the other hand, co-engagement of the activation or coactivation molecules of NK cells simultaneously with that of FcγRIIIa with infliximab (but not with 3G8) led to quantitative and qualitative variations in the functional responses of these cells.

### REFERENCES

Alter, G., Malenfant, J. M., and Altfeld, M. (2004). CD107a as a functional marker for the identification of natural killer cell activity. J Immunol Methods 294, 15-22.
Andre, P., Castriconi, R., Espeli, M., Anfossi, N., Juarez, T., Hue, S., Conway, H., Romagne, F., Dondero, A., Nanni, M., et al. (2004). Comparative analysis of human NK cell activation induced by NKG2D and natural cytotoxicity receptors. Eur J Immunol 34, 961-971.
Anegon I., Cuturi, M. C., Trinchieri, G., and Perussia, B. (1988). Interaction of Fc receptor (CD16) ligands induces transcription of interleukin 2 receptor (CD25) and lymphokine genes and expression of their products in human natural killer cells. J Exp Med 167, 452-472.
Atkinson, E. A., Gerrard, J. M., Hildes, G. E., and Greenberg, A. H. (1990). Studies of the mechanism of natural killer (NK) degranulation and cytotoxicity. J Leukoc Biol 47, 39-48.
Betts, M. R., Brenchley, J. M., Price, D. A., De Rosa, S. C., Douck, D. C., Roederer, M., and Koup, R. A. (2003). Sensitive and viable identification of antigen-specific CD8+ T cells by a flow cytometric assay for degranulation. J Immunol Methods 281, 65-78.
Betts, M. R., Price, D. A., Brenchley, J. M., Lore, K., Guenaga, F. J., Smed-Sorensen, A., Ambrozak, D. R., Migueles, S. A., Connors, M., Roederer, M., et al. (2004). The functional profile of primary human antiviral CD8+ T cell effector activity is dictated by cognate peptide concentration. J Immunol 172, 6407-6417.
Boehm, U., Klamp, T., Groot, M., and Howard, J. C. (1997). Cellular responses to interferon-gamma. Annu Rev Immunol 15, 749-795.
Borrego, F., Lopez-Beltran, A., Pena, J., and Solana, R. (1994). Downregulation of Fc gamma receptor 111A alpha (CD16-II) on natural killer cells induced by anti-CD16 mAb is independent of protein tyrosine kinases and protein kinase C. Cell Immunol 158, 208-217.
Brunner, K. T., Mauel, J., Cerottini, J. C., and Chapuis, B. (1968). Quantitative assay of the lytic action of immune lymphoid cells on 51-Cr-labelled allogeneic target cells in vitro; inhibition by isoantibody and by drugs. Immunology 14, 181-196.
Bryceson, Y. T., March, M. E., Ljunggren, H. G., and Long, E. O. (2006). Synergy among receptors on resting NK cells for the activation of natural cytotoxicity and cytokine secretion. Blood 107, 159-166.
Cartron, G., Dacheux, L., Salles, G., Solal-Celigny, P., Bardos, P., Colombat, P., and Watler, H. (2002). Therapeutic activity of humanized anti-CD20 monoclonal antibody and polymorphism in IgG Fc receptor FcgammaRIIIa gene. Blood 99, 754-758.
Colucci, F., Rosmaraki, E., Bregenholt, S., Samson, S. I., Di Bartolo, V., Turner, M., Vanes, L., Tybulewicz, V., and Di Santo, J. P. (2001). Functional dichotomy in natural killer cell signaling; Vavl-dependent and -independent mechanisms. J Exp Med 193, 1413-1424.
Cooley, S., Burns, L. J., Repka, T., and Miller, J. S. (1999). Natural killer cell cytotoxicity of breast cancer targets is enhanced by two distinct mechanisms of antibody-dependent cellular cytotoxicity against LFA-3 and HER2/neu. Exp Hematol 27, 1533-1541.
Cooper, M. A., Fehniger, T. A., Turner, S. C., Chen, K. S., Ghaheri, B. A., Ghayur, T., Carson, W. E., and Caligiuri, M. A. (2001). Human natural killer cells: a unique innate immunoregulatory role for the CD56(bright) subset. Blood 97, 3146-3151.
Dall'Ozzo, S., Tartas, S., Paintaud, G., Cartron, G., Colombat, P., Bardos, P., Watier, H., and Thibault, G. (2004). Rituximab-dependent cytotoxicity by natural killer cells: influence of FCGR3A polymorphism on the concentration-effect relationship. Cancer Res 64, 4664-4669.
Faroudi, M., Utzny, C., Salio, M., Cerundolo, V., Guiraud, M., Muller, S., and Valitutti, S. (2003). Lytic versus stimulatory synapse in cytotoxic T lymphocyte/target cell interaction: manifestation of a dual activation threshold. Proc Natl Acad Sci USA 100, 14145-14150.
Fukuda, M. (1991). Lysosomal membrane glycoproteins. Structure, biosynthesis, and intracellular trafficking. J Biol Chem 266, 21327-21330.
Gajewski, T. F., Goldwasser, E., and Fitch, F. W. (1988). Anti-proliferative effect of IFN-gamma in immune regulation. II. IFN-gamma inhibits the proliferation of murine bone marrow cells stimulated with IL-3, IL-4, or granulocyte-macrophage colony-stimulating factor. J Immunol 141, 2635-2642.
Greenwood, J., Clark, M., and Waldmann, H. (1993). Structural motifs involved in human IgG antibody effector functions. Eur J Immunol 23, 1098-1104.
Hesslein, D. G., Takaki, R., Hermiston, M. L., Weiss, A., and Lanier, L. L. (2006). Dysregulation of signaling pathways in CD45-deficient NK cells leads to differentially regulated cytotoxicity and cytokine production. Proc Natl Acad Sci USA 103, 7012-7017.
Huntington, N. D., Xu, Y., Nutt, S. L., and Tarlinton, D. M. (2005). A requirement for CD45 distinguishes Ly49D-mediated cytokine and chemokine production from killing in primary natural killer cells. J Exp Med 201, 1421-1433.
Kurago, Z. B., Lutz, C. T., Smith, K. D., and Colonna, M. (1998). NK cell natural cytotoxicity and IFN-gamma production are not always coordinately regulated: engagement of DX9 KIR+NK cells by HLA-B7 variants and target cells. J Immunol 160, 1573-1580.
Mendes, R., Bromelow, K. V., Westby, M., Galea-Lauri, J., Smith, I. E., O′Brien, M. E., and Souberbielle, B. E. (2000). Flow cytometric visualisation of cytokine production by CD3-CD56+NK cells and CD3+ CD56+NK-T cells in whole blood. Cytometry 39, 72-78.
Natarajan, K., Dimasi, N., Wang, J., Mariuzza, R. A., and Margulies, D. H. (2002). Structure and function of natural killer cell receptors: multiple molecular solutions to self, nonself discrimination. Annu Rev Immunol 20, 853-885.
Nathan, C. F., Prendergast, T. J., Wiebe, M. E., Stanley, E. R., Platzer, E., Remold, H. G., Welte, K., Rubin, B. Y., and Murray, H. W. (1984). Activation of human macrophages. Comparison of other cytokines with interferon-gamma. J Exp Med 160, 600-605.
Parihar, R., Dierksheide, J., Hu, Y., and Carson, W. E. (2002). IL-12 enhances the natural killer cell cytokine response to Ab-coated tumor cells. J Clin Invest 110, 983-992.
Pende, D., Cantoni, C., Rivera, P., Vitale, M., Castriconi, R., Marcenaro, S., Nanni, M., Biassoni, R., Bottino, C., Moretta, A., and Moretta, L. (2001). Role of NKG2D in tumor cell lysis mediated by human NK cells: cooperation with natural cytotoxicity receptors and capability of recognizing tumors of nonepithelial origin. Eur J Immunol 31, 1076-1086.
Rajagopalan, S., Fu, J., and Long, E. O. (2001). Cutting edge: induction of IFN-gamma production but not cytotoxicity by the killer cell Ig-like receptor KIR2DL4 (CD158d) in resting NK cells. J Immunol 167, 1877-1881.
Reff, M. E., Carner, K., Chambers, K. S., Chinn, P. C., Leonard, J. E., Raab, R., Newman, R. A., Hanna, N., and Anderson, D. R. (1994). Depletion of B cells in vivo by a chimeric mouse human monoclonal antibody to CD20. Blood 83, 435-445.
Shields, R. L., Namenuk, A. K., Hong, K., Meng, Y. G., Rae, J., Briggs, J., Xie, D., Lai, J., Stadlen, A., Li, B., et al. (2001). High resolution mapping of the binding site on human IgG1 for Fc gamma RI, Fc gamma RII, Fc gamma RIII, and FcRn and design of IgG1 variants with improved binding to the Fc gamma R. J Biol Chem 276, 6591-6604.
Shinkawa, T., Nakamura, K., Yamane, N., Shoji-Hosaka, E., Kanda, Y., Sakurada, M., Uchida, K., Anazawa, H., Satoh, M., Yamasaki, M., et al. (2003). The absence of fucose but not the presence of galactose or bisecting N-acetylglucosamine of human IgG1 complex-type oligosaccharides shows the critical role of enhancing antibody-dependent cellular cytotoxicity. J Biol Chem 278, 3466-3473.
Smyth, M. J., Cretney, E., Kelly, J. M., Westwood, J. A., Street, S. E., Yagita, H., Takeda, K., van Dommelen, S. L., Degli-Esposti, M. A., and Hayakawa, Y. (2005). Activation of NK cell cytotoxicity. Mol Immunol 42, 501-510.
Trinchieri, G. (1989). Biology of natural killer cells. Adv Immunol 47, 187-376.
Umana, P., Jean-Mairet, J., Moudry, R., Amstutz, H., and Bailey, J. E. (1999). Engineered glycoforms of an antineuroblastoma IgG1 with optimized antibody-dependent cellular cytotoxic activity. Nat Biotechnol 17, 176-180.
Vitale, M., Caruso, A., Licenziati, S., Rodella, L., Fiorentini, S., Zauli, G., Castelli, F., Manzoli, F. A., and Turano, A. (2000). Differential production of IFN-gamma, analyzed at the single-cell level, by specific subsets of human NK and T cells from healthy and HIV(+) subjects. Cytometry 39, 189-194.
Wing, M. G., Moreau, T., Greenwood, J., Smith, R. M., Hale, G., Isaacs, J., Waldmann, H., Lachmann, P. J., and Compston, A. (1996). Mechanism of first-dose cytokine-release syndrome by CAMPATH 1-H: involvement of CD16 (FcgammaRIII) and CD11a/CD18 (LFA-1) on NK cells. J Clin Invest 98, 2819-2826.

1. A method for evaluating, in vitro, the effector functions of cells expressing FcγRIIIa in response to a test monoclonal antibody, comprising:
(i) the cells expressing FcγRIIIa are brought into contact with said monoclonal antibody, which is fixed on a support, in the presence of an agent for inhibiting the secretion of cytokines by said cells;
(ii) by way of positive control for the activation of the cells expressing FcγRIIIa, the same experiment is carried out using, in place of the test monoclonal antibody, a monoclonal antibody directed against the FcγRIIIa receptor; and
(iii) after an incubation period of at least 1 hour, the response of the cells expressing FcγRIIIa is observed by measuring the presence of the CD107 marker at the cell surface, and also the presence of intracellular interferon gamma (IFNγ) and/or of intracellular tumor necrosis factor alpha (TNFα).

2. The method as claimed in claim 1, wherein the cells expressing FcγRIIIa are NK cells or T lymphocytes.

3. The method as claimed in claim 1, wherein the presence of the CD107 marker at the cell surface, and the intracellular IFNγ and/or the intracellular TNFα, are measured by flow cytometry.

4. The method as claimed in claim 1, wherein the incubation is carried out in the presence of an anti-CD107 monoclonal antibody coupled to a label.

5. The method as claimed in claim 1, wherein the interferon gamma measurement is carried out by permeabilizing the cells at the end of the incubation, and then by using an anti-IFNγ monoclonal antibody coupled to a label.

6. The method as claimed in claim 1, wherein the TNF alpha measurement is carried out by permeabilizing the cells at the end of the incubation, and then by using an anti-TNFα monoclonal antibody coupled to a label.

7. The method as claimed in claim 1, comprising, in addition, a step of measuring the expression of FcγRIIIa/CD16a at the surface of the cells, by flow cytometry after labeling said cells, at the end of the incubation, with an anti-CD 16 monoclonal antibody coupled to a label.

8. The method as claimed in claim 7, wherein the labels coupled to the anti-CD107, anti-IFNγ, and anti-TNFα monoclonal antibodies are distinct fluorochromes.

9. The method as claimed in claim 1, wherein the agent for inhibiting the secretion of cytokines by said cells expressing FcγRIIIa comprises Brefeldin A.

10. The method as claimed in claim 1, wherein the test monoclonal antibody and the monoclonal antibody directed against the FcγRIIIa receptor used as control are bound to plates, by incubating said plates with a concentration of approximately 1 to 3 μg/ml of said antibodies, for at least 6 h.

11. The method as claimed in claim 1, wherein the test monoclonal antibody and the monoclonal antibody directed against the FcγRIIIa receptor used as control are bound to plates, by incubating said plates with a concentration of approximately 0.01 to 1 μg/ml of said antibodies, for at least 6 h.

12. The method as claimed in claim 1, wherein the period of incubation of the cells expressing FcγRIIIa with the monoclonal antibodies is at least 1 hour.

13. The method as claimed in claim 1, comprising, in addition, by way of positive control for the response capacity of the cells expressing FcγRIIIa, a step (iia) of stimulating said cells with a mixture of a phorbol ester and of a calcium ionophore.

14. The method as claimed in claim 1, further comprising after the end of the incubation, the following steps:
(iv) the cells expressing FcγRIIIa are labeled with an anti-CD16 antibody coupled to a label,
(v) the cells expressing FcγRIIIa are permeabilized, and then labeled with an anti-IFNγ monoclonal antibody coupled to a label, and/or with an anti-TNFα monoclonal antibody coupled to a label; and,
(vi) the cells thus labeled are analyzed by flow cytometry.

15. The method as claimed in claim 2, wherein the NK cells and/or the T lymphocytes brought into contact with the monoclonal antibodies have been isolated from one or more blood samples.

16. The method as claimed in claim 15, wherein the cells brought into contact with the monoclonal antibodies are purified NK cells and/or purified T lymphocytes.

17. The method as claimed in claim 1, wherein steps (i) and (ii) are carried out with one or more samples of peripheral blood mononuclear cells (PBMCs) and/or of peripheral blood lymphocytes (PBLs) and/or of whole blood.

18. The method as claimed in claim 17, wherein the cells are labeled with an anti-CD56 monoclonal antibody coupled to a label, before the step of analysis by flow cytometry.

19. The method as claimed in claim 2, wherein, in steps (i) and (ii), the NK cells are costimulated with antibodies directed against one or more of the receptors chosen from NKp30, NKp46, NKG2D, 2B4, CD2, CD45, DNAM and CD11a.

20. The method as claimed in claim 19, wherein the antibodies used for the costimulation are monoclonal antibodies adsorbed onto a support.

21. A method for predicting the response of a patient to a treatment with a given monoclonal antibody comprising quantifying the effector functions of cells expressing FcγRIIIa in the patient in response to the given monoclonal antibody by measuring the presence of the CD107 marker at the cell surface of cells expressing FcγRIIIa, and also the presence of intracellular interferon gamma (IFNγ) and/or of intracellular tumor necrosis factor alpha (TNFα).

22. The method as claimed in claim 21, wherein the method is carried out with cells expressing FcγRIIIa which come from the patient.

23. A method, for comparing the ability of different recombinant monoclonal antibodies to induce antibody-dependent cellular cytotoxicity (ADCC) and antibody-dependent cytokine production (ADCP) by cells expressing FcγRIIIa comprising comparing the presence of the CD107 marker at the cell surface of cells expressing FcγRIIIa, and also the presence of intracellular interferon gamma (IFNγ) and/or of intracellular tumor necrosis factor alpha (TNFα).

24. A method for improving the effectiveness of recombinant monoclonal antibodies for therapeutic purposes comprising determining the presence of the CD107 marker at the cell surface of cells expressing FcγRIIIa, and also the presence of intracellular interferon gamma (IFNγ) and/or of intracellular tumor necrosis factor alpha (TNFα).

25. A method for carrying out a quality control of a sample of recombinant monoclonal antibodies comprising determining the presence of the CD107 marker at the cell surface of cells expressing FcγRIIIa, and also the presence of intracellular interferon gamma (IFNγ) and/or of intracellular tumor necrosis factor alpha (TNFα).

26. The method as claimed in claim 23, wherein the method is carried out with cells expressing FcγRIIIa which come from healthy individuals and/or from patients.

27. The method as claimed in claim 23, wherein the method is carried out with one or more NK cell and/or T lymphocyte lines.

28. A kit that can be used for carrying out a method as claimed in claim 1, comprising at least the following elements:
an anti-CD16 monoclonal antibody coupled to a label,
an anti-IFNγ monoclonal antibody coupled to a label and/or an anti-TNFα monoclonal antibody coupled to a label, and
a plate comprising at least one well coated with a monoclonal antibody directed against the FcγRIIIa receptor.

29. The method as claimed in claim 12, wherein the period of incubation of the cells expressing FcγRIIIa with the monoclonal antibodies is 2 to 4 hours.
